SEQUENCE LISTING (14 sequences)

SEQ ID NO: 1 - protein, 242 aa, Homo sapiens
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 107 aa, Homo sapiens
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 3 - protein, 120 aa, Homo sapiens
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 4 - protein, 246 aa, Homo sapiens
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGGGGSGGGGSSGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 5 - protein, 113 aa, Homo sapiens
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 6 - protein, 118 aa, Homo sapiens
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 7 - protein, 244 aa, Homo sapiens
DIVLTQPPSVSGAPGQRVTISCSGSESNIGNNYVQWYQQLPGTAPKLLIYDNNQRPSGVPDRFSGSKSGTSASLAITGLQSEDEADYYCQSYDMNVHVFGGGTKLTVLGGGGGSGGGGSGGGGSQVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCARSPRYRGAFDYWGQGTLVTVSS

SEQ ID NO: 8 - protein, 109 aa, Homo sapiens
DIVLTQPPSVSGAPGQRVTISCSGSESNIGNNYVQWYQQLPGTAPKLLIYDNNQRPSGVPDRFSGSKSGTSASLAITGLQSEDEADYYCQSYDMNVHVFGGGTKLTVLG

SEQ ID NO: 9 - protein, 120 aa, Homo sapiens
QVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCARSPRYRGAFDYWGQGTLVTVSS

SEQ ID NO: 10 - protein, 5 aa, synthetic construct
GGGGS

SEQ ID NO: 11 - protein, 4 aa, synthetic construct
GGGS

SEQ ID NO: 12 - protein, 5 aa, synthetic construct
SGGGG

SEQ ID NO: 13 - protein, 4 aa, synthetic construct
SGGG

SEQ ID NO: 14 - protein, 15 aa, synthetic construct
RGRGRGRGRSRGGGS

(19) United States
(12) Patent Application Publication
(10) Pub. No.: US 2026/0115290 A1
(43) Pub. Date: Apr. 30, 2026

(54) INHIBITORY CHIMERIC ANTIGEN RECEPTOR AND USES THEREOF

(71) Applicant: University of Southern California, Los Angeles, CA (US)

(72) Inventors: Jianming Xie, Los Angeles, CA (US); Liang Rong, Los Angeles, CA (US); Fan Fei, Los Angeles, CA (US)

(73) Assignee: University of Southern California, Los Angeles, CA (US)

(21) Appl. No.: 18/702,150

(22) PCT Filed: Oct. 27, 2022
(86) PCT No.: PCT/US2022/078782

§ 371 Date: Apr. 17, 2024

Related U.S. Application Data
(60) Provisional application No. 63/272,785, filed on Oct. 28, 2021.

(51) Int. Cl. A61K 40/15; A61K 40/31; A61K 40/42; A61P 35/00; C07K 14/725; C07K 16/28

(52) U.S. Cl. CPC A61K 40/15; A61K 40/31; A61K 40/421; A61K 40/4211; A61P 35/00; C07K 14/7051; C07K 16/2803; C07K 16/2833; A61K 2239/22; A61K 2239/23; A61K 2239/28; C07K 2317/622; C07K 2317/76; C07K 2319/03

(57) ABSTRACT

We provide compositions and methods to enhance the anti-cancer specificity of chimeric antigen receptor natural killer cells (CAR-NK) by activating them against cancer antigens while inhibiting them against human leukocyte antigen DR (HLA-DR). HLA-DR is reportedly lost or downregulated in a substantial proportion of hematologic malignancies. An anti-HLA-DR inhibitory CAR (iCAR) is provided to effectively suppress NK cell activation against HLA-DR-expressing cells. Dual CAR-NK cells, which co-express the anti-CD19 or anti-CD33 activating CAR and the anti-HLA-DR iCAR, can preferentially target HLA-DR-negative cells over HLA-DR-positive cells in vitro. The HLA-DR-mediated inhibition is positively correlated with both iCAR and HLA-DR densities. Surrounding cells that express HLA-DR do not affect the target selectivity of the dual CAR-NK cells. We have confirmed that HLA-DR-positive cells are resistant to dual CAR-NK cell-mediated killing in a xenograft mouse model. This can be used for enhancing CAR-NK and CAR-T cell specificity against malignancies with HLA-DR loss.

### CROSS-REFERENCE TO RELATED APPLICATIONS

This application includes a claim of priority to U.S. provisional patent application No. 63/272,785, filed Oct. 28, 2021, the entirety of which is hereby incorporated by reference.

### REFERENCE TO SEQUENCE LISTING

This application contains a Sequence Listing submitted as an electronic file named “065715_000135WOPT_SEQ LISTING_ST26”, having a size in bytes of 14,542 bytes, and created on Oct. 27, 2022. The information contained in this electronic file is hereby incorporated by reference in its entirety.

### FIELD OF INVENTION

This invention relates to cell therapies based on chimeric antigen receptors.

### BACKGROUND

Genetic engineering of T cells and natural killer (NK) cells with chimeric antigen receptors (CAR) has emerged as a powerful new therapeutic approach for cancer and, in particular, hematologic malignancies. However, most target antigens are not cancer-specific but instead are also expressed on normal cells (albeit sometimes at lower levels). As such, CAR-T and CAR-NK cells can mistakenly attack normal cells—a severe adverse effect known as on-target off-tumor toxicity. For example, anti-CD19 CAR-T cell therapy against B-cell leukemia and lymphoma leads to unwanted depletion of normal B cells with prolonged B cell aplasia. Likewise, anti-CD33 CAR-T cells, which have shown potent cytotoxicity to acute myeloid leukemia (AML), can attack healthy myeloid cells in the blood and bone marrow, inducing severe cytopenias. Clearly, there is an urgent need to enhance the specificity of CAR-T and CAR-NK cells against cancer.

While cancer-specific surface antigens are rarely available, cancer cells often lose or downregulate the expression of human leukocyte antigens (HLA) as a potential mechanism of escape from immune surveillance. For example, HLA-DR, the most abundant HLA class II molecule, is reportedly absent or downregulated in up to 33% of all cases of diffuse large B-cell lymphoma (DLBCL) and over 50% of those arising in immune-privileged sites such as the central nervous system and the testis. HLA-DR loss is also found in other types of hematologic malignancies, including approximately 15-17% of AML, 40% of classical Hodgkin lymphoma (CHL), 23% of chronic myelomonocytic leukemia (CMML), and some cases of chronic myeloid leukemia (CML). As an immune escape mechanism, HLA-DR loss is frequently correlated with lower T cell infiltration and reduced patient survival. Importantly, loss of HLA antigen expression is also recognized as a resistance mechanism associated with post-transplant relapses after allogeneic hematopoietic stem cell transplantation (allo-HSCT), a curative therapy for all types of hematologic malignancies. Two recent studies reported that up to 50% of AML patients with post-transplant relapse are associated with complete or partial loss of HLA-DR and other HLA class II molecules. Thus, a substantial proportion of hematologic malignancies are HLA-DR-deficient at diagnosis and/or relapse.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a genetically engineered immune cell expressing a first chimeric antigen receptor (CAR) and a second, inhibitory CAR, by introduction of one or more genes, wherein the first CAR comprises: a first antigen-specific binding domain, a first transmembrane domain, and a first intracellular domain; and the second, inhibitory CAR comprises: a second antigen-specific binding domain, which is specific for and binds a human leukocyte antigen (HLA), a second transmembrane domain, and a second intracellular domain, which is a signaling domain or intracellular tail of an immunoinhibitory receptor.

In various embodiments, the genetically engineered immune cell can be a natural killer (NK) cell or a T cell, and the genetically engineered immune cell is more selective in targeting a malignant cell or tumor cell that expresses an antigen that the first antigen-specific binding domain binds to but does not express the HLA, than targeting a malignant cell or tumor cell that expresses both the antigen that the first antigen-specific binding domain binds to and the HLA.

In various embodiments, the second antigen-specific binding domain can be specific for and binds HLA-DR, and the genetically engineered immune cell is an NK cell that does not express HLA-DR.

In various embodiments, the second antigen-specific binding domain can comprise: a single-chain variable fragment (scFv) having the sequence as set forth in SEQ ID NO:7 or at least 90% identical to SEQ ID NO:7, or a variable region of the light chain (VL) having the sequence as set forth in SEQ ID NO:8 or at least 90% identical to SEQ ID NO:8, and a variable region of the heavy chain having the sequence as set forth in SEQ ID NO:9 or at least 90% identical SEQ ID NO:9, and a linker between the VL and the VH.

In various embodiments, the second antigen-specific binding domain can comprise: a single-chain variable fragment (scFv) from anti-HLA-DR monoclonal antibody clone L243.

In various embodiments, the second intracellular domain can be the signaling domain or intracellular tail of an immunoinhibitory receptor selected from the group consisting of programmed cell death protein 1 (PD-1), cytotoxic T lymphocyte-associated antigen-4 (CTLA-4), lymphocyte-activation gene 3 (LAG-3), 2B4, B- and T-lymphocyte attenuator (BTLA), and a combination thereof.

In various embodiments, the second intracellular domain can be the signaling domain or intracellular tail of PD-1.

In various embodiments, the first antigen-specific binding domain can be specific for and binds an antigen selected from the group consisting of antigens specific for cancer, an inflammatory disease, a neuronal disorder, diabetes, a cardiovascular disease, an infectious disease, an autoimmune disease, and a combination thereof.

In various embodiments, the first antigen-specific binding domain can be specific for and binds CD33.

In various embodiments, the first antigen-specific binding domain can comprise: a single-chain variable fragment (scFv) having the sequence as set forth in SEQ ID NO:4 or at least 90% identical to SEQ ID NO:4, or a variable region of the light chain (VL) having the sequence as set forth in SEQ ID NO:5 or at least 90% identical to SEQ ID NO:5, and a variable region of the heavy chain having the sequence as set forth in SEQ ID NO:6 or at least 90% identical SEQ ID NO:6, and a linker between the VL and the VH.

In various embodiments, the first antigen-specific binding domain can be specific for and binds CD19.

In various embodiments, the first antigen-specific binding domain can comprise: a single-chain variable fragment (scFv) having the sequence as set forth in SEQ ID NO:1 or at least 90% identical to SEQ ID NO:1, or a variable region of the light chain (VL) having the sequence as set forth in SEQ ID NO:2 or at least 90% identical to SEQ ID NO:2, and a variable region of the heavy chain (VH) having the sequence as set forth in SEQ ID NO:3 or at least 90% identical SEQ ID NO:3, and a linker between the VL and the VH.

In various embodiments, the first transmembrane domain can comprise CD28 transmembrane domain, CD8 transmembrane domain, a transmembrane domain of a zeta chain of a T cell receptor complex, or a combination thereof; and wherein the second transmembrane domain comprises a transmembrane domain of the immunoinhibitory receptor, CD28 transmembrane domain, CD8 transmembrane domain, a transmembrane domain of a zeta chain of a T cell receptor complex, or a combination thereof.

In various embodiments, the first CAR further can comprise a first extracellular spacer domain, positioned between the first antigen-specific binding domain and the first transmembrane domain, and the first extracellular spacer domain comprises (i) a hinge region of CD8α, (ii) a hinge region of IgG4, (iii) a hinge and CH2 region of IgG4, (iv) a hinge, CH2 and CH3 region of IgG4, (v) a hinge, CH2 and CH3 region of IgG1, (vi) a hinge region of IgG1, (vi) a hinge and CH2 region of IgG1, or (vii) a combination of any two or more of (i)-(vi); and/or wherein the second, inhibitory CAR further comprises a second extracellular spacer domain, positioned between the second antigen-specific binding domain and the second transmembrane domain, and the second extracellular spacer domain comprises (1) an extracellular topological domain of the immunoinhibitory receptor, (2) a hinge region of CD8α, (3) a hinge region of IgG4, (4) a hinge and CH2 region of IgG4, (5) a hinge, CH2 and CH3 region of IgG4, (6) a hinge, CH2 and CH3 region of IgG1, (7) a hinge region of IgG1, (8) a hinge and CH2 region of IgG1, or (9) a combination of any two or more of (1)-(8).

In various embodiments, the first intracellular domain can comprise one or more of a CD3 zeta chain, FcγRIII, FcεRI, a cytoplasmic tail of a Fc receptor, and an immunoreceptor tyrosine-based activation motif (ITAM) bearing cytoplasmic receptors; and optionally further comprising a co-stimulatory domain selected from the group consisting of CD28, CD137 (4-1BB), CD134 (OX40), Dap10, CD27, CD2, CD5, ICAM-1, LFA-1, Lck, TNFR-I, TNFR-II, Fas, CD30, CD40 and a combination thereof.

In various embodiments, the first CAR, the second, inhibitory CAR, or both, can further comprise a protein tag at N terminus; and the protein tag of the first CAR and that of the second, inhibitory CAR are different, if both are present.

In various embodiments, the genetically engineered immune cell can have a secretion level of IFN-γ, after contact of the genetically engineered immune cell with a target cell expressing an antigen that the first antigen-specific binding domain is specific for and binds to, and/or have a cytotoxicity level towards the target cell expressing the antigen that the first antigen-specific binding domain is specific for and binds to, is statistically similar to that of a genetically engineered immune cell that is otherwise identical except for lacking the second, inhibitory CAR.

In various embodiments, the genetically engineered immune cell can have a secretion level of IFN-γ after the contact with the target cell, and/or have degranulation if the genetically engineered immune cell is an NK cell upon incubation with a second target cell expressing the HLA, and/or whose cytotoxicity level towards the second target cell expressing the HLA, is at least 80%, 70%, 60%, 50%, 40%, 30%, or 20% lower, or at least two-fold lower, than the genetically engineered immune cell that is otherwise identical except for lacking the second, inhibitory CAR.

In various embodiments, the second, inhibitory CAR can be encoded by a coding sequence in a gene construct introduced to the genetically engineered immune cell, and the second, inhibitory CAR is operably linked to a transcriptional promoter comprising UBC1, EF1α or MSCV.

Various embodiments of the present invention provide for a combination, comprising: a first genetically engineered immune cell expressing a first chimeric antigen receptor (CAR), wherein the first CAR comprises: a first antigen-specific binding domain, a first transmembrane domain, and a first intracellular domain; and a second genetically engineered immune cell expressing a second, inhibitory CAR, wherein the second, inhibitory CAR comprises: a second antigen-specific binding domain, which is specific for and binds a human leukocyte antigen (HLA), a second transmembrane domain, and a second intracellular domain, which is a signaling domain or intracellular tail of an immunoinhibitory receptor.

Various embodiments of the present invention provide for a genetically engineered natural killer (NK) cell expressing an inhibitory chimeric antigen receptor (CAR) by introduction of a gene, wherein the inhibitory CAR comprises: an antigen-specific binding domain, which is specific for and binds human leukocyte antigen DR (HLA-DR), a transmembrane domain, and an intracellular domain, which is a signaling domain or intracellular tail of an immunoinhibitory receptor of PD-1.

Various embodiments of the present invention provide for a vector encoding the second, inhibitory CAR of the present invention as described herein.

Various embodiments of the present invention provide for a method of preparing a genetically engineered immune cell of the present invention as described herein, comprising introducing to an immune cell a first vector encoding the first CAR and a second vector encoding the second, inhibitory CAR.

In various embodiments, the first vector and the second vector can be introduced sequentially.

In various embodiments, the method can further comprise selecting the immune cell expressing the first CAR after the first vector is introduced to the immune cell, wherein the second vector is introduced to the selected immune cell expressing the first CAR, so as to produce the genetically engineered immune cell expressing both the first CAR and the second, inhibitory CAR.

Various embodiments of the present invention provide for a method of treating a subject with a hematologic malignancy or a solid tumor, comprising: administering to the subject a pharmaceutical composition comprising an effective quantity of genetically engineered immune cells of the present invention as described herein.

In various embodiments, the subject can be detected or diagnosed with complete or partial loss of HLA-DR in the hematologic malignancy, or wherein the subject is detected or diagnosed with complete or partial loss of HLA-A, HLA-B and/or HLA-C in the solid tumor.

In various embodiments, the subject can have a hematologic malignancy, and the hematologic malignancy comprises acute myeloid leukemia (AML), classical Hodgkin lymphoma (CHL), chronic myelomonocytic leukemia (CMML), chronic myeloid leukemia (CML), or a combination thereof.

In various embodiments, the subject can be an AML patient with post-transplant relapse.

In various embodiments, the pharmaceutical composition can be administered to bloodstream or bone marrow of the subject.

In various embodiments, the genetically engineered immune cells can be produced by introducing the one or more genes encoding the first CAR and the second, inhibitory CAR into allogeneic NK cells obtained from another subject.

In various embodiments, the method can further comprise comprising administering to the subject a T-cell receptor-based immunotherapy or donor lymphocyte infusion, so as to target cancer cells that express the HLA.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1. Schematic illustration of an anti-HLA-DR iCAR to reduce on-target off-tumor toxicity. The dual CAR-NK cell expresses an anti-CD19 CAR and an anti-HLA-DR iCAR. The cancer cell, which expresses CD19 but not HLA-DR, would be killed. The normal cell, which expresses both CD19 and HLA-DR, would inhibit CAR-NK cell-mediated cytotoxicity via engaging the anti-HLA-DR iCAR. The scheme is also applicable if CD33 (or any other cancer antigen) replaces CD19.

FIGS. 2A-2B. Engineering NK-92MI cells to express an anti-CD19 CAR with or without an anti-HLA-DR iCAR. (2A) Schematic design of the anti-CD19 CAR and the anti-HLA-DR iCAR. (2B) Flow cytometric analysis of untransduced and transduced NK-92MI cells. The CD19 CAR and the HLA-DR iCAR were stained with a PE-labeled anti-HA tag antibody and an APC-labeled anti-FLAG tag antibody, respectively. Data are representative of three independent experiments.

FIG. 2C. Flow cytometric analysis of NK, single CAR-NK, and dual CAR-NK cells. Cells were stained for CD19 CAR, HLA-DR iCAR, CD19, HLA-DR, CD56, PD-1, and LAG-3. Negative controls were NK cells stained with isotype control antibodies.

FIG. 3A-3B. Verification of the expression of CD19 and HLA-DR on the surface of target cells. Flow cytometric analysis of CD19 (3A) and HLA-DR (3B) on the cell surface of six cell lines. Cells were stained with PE-conjugated anti-CD19 and anti-HLA-DR antibodies, respectively. Isotype antibodies were used as negative controls. Images are representative of three independent experiments with similar results.

FIG. 4A-4C. Dual CAR-NK cells preferentially recognize and kill HLA-DR-negative cells over HLA-DR-positive cells in vitro. (4A) Comparison of IFN-γ production by single and dual CAR-NK cells in response to different target cells. Single and dual CAR-NK cells were incubated with each of the six target cells (K562, K562- CD19, K562-CD19-HLA-DR, KOPN1, Nalm6, and Raji) at an E:T ratio of 1:1 for 4 hours at 37° C. The cell culture supernatant was collected, and the concentration of IFN-γ was measured by ELISA. Data are shown as mean±SEM of triplicates. (4B) Comparison of CD107a expression by single and dual CAR-NK cells in response to different target cells. Single and dual CAR-NK cells were incubated with each of the six target cells for 1 hour at 37° C., in the presence of a PE-conjugated anti-CD107a antibody. Cells were then treated with monensin (GolgiStop), incubated for 4 hours, stained with an anti-CD56 antibody, and analyzed by flow cytometry. The percentage of CD107a+ cells was determined, which indicates the level of degranulation. Unmodified NK-92MI cells were used as a negative control. (4C) Comparison of the cytotoxicity of single and dual CAR-NK cells against different target cells. The percentage of cytotoxicity was measured by LDH release. Unmodified NK-92MI cells were used as the negative control. Data are shown as mean±SEM of three independent experiments. Statistical significance is calculated by unpaired two-tailed Student's t-test. **** p<0.0001; *** p<0.001; ** p<0.01; * p<0.05; ns: not significant.

FIG. 4D. Flow cytometric analysis of CD69 expression on NK and CAR-NK cells. NK, single CAR-NK, and dual CAR-NK cells were cocultured with K562-CD19, KOPN1, Nalm6 cells for 4 h at 37° C. Cells were stained with a PE-Cy7-labeled anti-CD69 antibody and an APC-labeled anti-CD56 antibody followed by flow cytometry analysis. Data are shown as mean±SEM of two independent experiments. Statistical significance is calculated by unpaired two-tailed Student's t-test. **** p<0.0001; *** p<0.001; ns: not significant.

FIG. 4E. Cytotoxicity of NK and CAR-NK cells against different target cells. NK, single CAR-NK, and dual CAR-NK cells were incubated with K562, K562-CD19, K562-CD19-HLA-DR, KOPN1, Nalm6, and Raji cells for 4 hours at three E:T ratios (0.2:1, 1:1, 5:1) for 4 hours. Cells were then stained with an APC-conjugated anti-CD56 antibody and aqua live/dead stain and subjected to flow cytometry analysis. The percentage of cytotoxicity was calculated as [(A−B)/A×100], in which A and B were the percentages of viable target cells (CD56-negative) in the control group and in the experimental group, respectively. Data are shown as mean±SEM of three independent experiments. Statistical significance is calculated by unpaired two-tailed Student's t-test. **** p<0.0001; *** p<0.001; ** p<0.01; * p<0.05; ns: not significant.

FIG. 5A-5C. The level of iCAR-mediated inhibition is dependent on the availability of HLA-DR on target cells and iCAR on effector cells. (5A) The HLA-DR antigens on K562-CD19-HLA-DR cells were blocked with different concentrations of anti-HLA-DR scFv. These cells were then cocultured with NK, single CAR-NK, and dual CAR-NK cells, respectively. After 4 hours, the IFN-γ level in the coculture supernatant was assessed by ELISA. Data are shown as mean±SEM of two independent experiments. (5B) Flow cytometric analysis of single CAR-NK cells and three dual CAR-NK cell populations expressing HLA-DR iCAR at different levels. Cells were stained with a PE-labeled anti-FMC63 scFv antibody (for CD19 CAR) and a PE-labeled anti-FLAG tag antibody (for HLA-DR iCAR) at saturating concentrations, respectively. Untransduced NK-92MI cells were used as the negative control. (5C) Comparison of IFN-γ production by single and the three dual CAR-NK cells against K562-CD19, KOPN1, or Nalm6 cells. Cells were incubated at a 1:1 E:T ratio for 4 hours. The concentrations of IFN-γ were measured by ELISA. Data are shown as mean±SEM of two independent experiments. Statistical significance is calculated by unpaired two-tailed Student's t-test. **** p<0.0001; *** p<0.001; ** p<0.01; * p<0.05; ns: not significant.

FIG. 5D. Purification and verification of recombinantly expressed anti-HLA-DR scFv. (Upper) Refolded protein was purified by gel filtration chromatography. The running buffer is 1×PBS buffer pH 7.4. (Lower) Peak fractions were analyzed by SDS-PAGE. Fractions 30-37 were pooled and concentrated to 1 mg/ml for subsequent use.

FIG. 5E. Characterization of dual CAR-NK cells with different levels of iCAR expression. (Top) Titration of the PE-labeled anti-FMC63 scFv antibody and the PE-labeled anti-FAG antibody for staining the CAR and the iCAR on CAR-NK cells, respectively. Dual CAR-NK (iCAR^{high}) cells were stained with each antibody at multiple different dilutions. The mean fluorescence intensities (MFI) of stained cells were measured by flow cytometry. The saturating concentration of each antibody was identified as the minimal concentration at which the MFI of stained cells reached a plateau. (Bottom) Comparison of the CAR and iCAR expression on the single CAR-NK cells and three dual CAR-NK cell populations. Cells were stained with the PE-labeled anti-FMC63 scFv antibody at a 1:5 dilution and the PE-labeled anti-FAG antibody at a 1:20 dilution, separately. The MFIs of cells stained with each antibody were measured by flow cytometry. Data were shown as mean±SEM of three independent experiments.

FIG. 6A-6D. The target selectivity of dual CAR-NK cells is not affected by HLA-DR-expressing surrounding cells. (6A) Flow cytometric analysis of KG-1 cells stained with PE-conjugated antibodies against CD19 and HLA-DR, separately. Isotype antibodies were used as the negative control. Images are representative of three independent experiments with similar results. (6B) ELISA analysis of IFN-γ production by single CAR-NK cells against KG-1 cells or K562-CD19 cells after a 4-hour incubation. Data are shown as mean±SEM of three independent experiments. (6C)-(6D) Comparison of the activation levels of single and dual CAR-NK cell activation against K562-CD19 cells (6C) or K562-CD19-HLA-DR cells (6D) in the presence or absence of KG-1 cells. CAR-NK cells, target cells, and surrounding KG-1 cells were cocultured at the indicated E:T:S ratios. After a 4-hour incubation, cell culture supernatants were collected to assess for IFN-γ secretion by ELISA. Data are shown as mean±SEM of three independent experiments. Statistical significance is calculated by unpaired two-tailed Student's t-test. **** p<0.0001; *** p<0.001; ** p<0.01; ns: not significant.

FIG. 6E. The target selectivity of dual CAR-NK cells is not affected by HLA-DR-expressing surrounding myeloid cells. (Top) Myeloid cells in human PBMC were enriched by immunomagnetic negative selection, followed by cell surface marker staining and flow cytometry. The expression or lack of HLA-DR and CD19 was also examined. Isotype antibodies were used as the negative control. Images are representative of three independent experiments with similar results. (Bottom left) ELISA analysis of IFN-γ production by single CAR-NK cells against myeloid cells or K562-CD19 cells after a 4-hour incubation. Data are shown as mean±SEM of three independent experiments. (Bottom middle)-(Bottom right) Comparison of the activation levels of single and dual CAR-NK cell activation against K562-CD19 cells (middle) or K562-CD19-HLA-DR cells (right) in the presence or absence of human myeloid cells. CAR-NK cells, target cells, and surrounding myeloid cells were cocultured at the indicated E:T:S ratios. After a 4-hour incubation, cell culture supernatants were collected to assess for IFN-D secretion by ELISA. Data are shown as mean±SEM of three independent experiments. Statistical significance is calculated by unpaired two-tailed Student's t-test. *** p<0.001; ** p<0.01; * p<0.05; ns: not significant.

FIG. 7A-7G. HLA-DR-positive cells, but not HLA-DR-negative cells, are resistant to dual CAR-NK cell-mediated cytotoxicity in vivo. (7A) Schematic diagram of the in vivo killing assay. NSG mice were inoculated with 5×105 K562-CD19-HLA-DR-Luc or K562-CD19-Luc cells through tail vein injection on day 0 and then treated with 1×107 NK cells, single CAR-NK cells, or dual CAR-NK cells through tail vein injection on day 3. Tumor growth was monitored by in vivo bioluminescence imaging on days 3, 10, 17, and 24. (7B) Bioluminescence images of K562-CD19-HLA-DR tumor growth in mice treated with NK cells (left), single CAR-NK cells (middle), and dual CAR-NK cells (right). (7C) Quantification of bioluminescence in each treatment group (n=5) on day 24. Data were shown as mean±SEM. Statistical significance is calculated by unpaired two-tailed Student's t-test. **** p<0.0001; ** p<0.01; ns: not significant. (7D) Survival of mice in each treatment group (n=5) were shown in Kaplan-Meier curves. Statistical significance was calculated by log-rank (Mantel-Cox) test. ** p<0.01; ns: not significant. (7E) Bioluminescence images of K562-CD19 tumor growth in mice treated with NK cells (left), single CAR-NK cells (middle), and dual CAR-NK cells (right). (7F) Quantification of bioluminescence in each treatment group (n=5) on day 24. Data were shown as mean±SEM. Statistical significance is calculated by unpaired two-tailed Student's t-test. * p<0.05; ns: not significant. (7G) Survival of mice in each treatment group (n=5) were shown in Kaplan-Meier curves. Statistical significance of survival data was calculated by log-rank (Mantel-Cox) test. ** p<0.01; ns: not significant.

FIG. 7H. Raji cells (CD19⁺HLA-DR⁺) are also resistant to dual CAR-NK cell-mediated cytotoxicity in vivo. At the top is a schematic diagram of the in vivo killing assay. NSG mice were inoculated with 5×10⁵ Raji-Luc cells through tail vein injection on day 0 and then treated with 1×10⁷ NK cells, single CAR-NK cells, or dual CAR-NK cells through tail vein injection on days 3, 5, and 7. Tumor growth was monitored by in vivo bioluminescence imaging on days 3, 10, and 17. Representative bioluminescence images were shown of tumor growth in mice treated with NK cells, with single CAR-NK cells, or with dual CAR-NK cells. The bar graph shows quantification of bioluminescence in each treatment group (n=5) on day 17. Data were shown as mean±SEM. Statistical significance is calculated by unpaired two-tailed Student's t-test. ** p<0.01. The line graph shows survival of mice in each treatment group (n=5) were shown in Kaplan-Meier curves. Statistical significance was calculated by log-rank (Mantel-Cox) test. ** p<0.01; ns: not significant.

FIG. 8A-8C. Anti-CD33 CAR-NK cells bearing the anti-HLA-DR iCAR preferentially target HLA-DR-negative AML cells. (8A) Flow cytometric analysis of NK cells, CD33-targeted single and dual CAR-NK cells. Cells were stained for flow cytometry with a PE-labeled anti-HA antibody and an APC-labeled anti-FLAG antibody to assess for CD33 CAR and HLA-DR CAR expression, respectively. (8B) IFN-γ production of CD33-targeted single and dual CAR-NK cells against HL-60 (CD33+HLA-DRneg) and KG-1 (CD33+HLA-DR+) cells. CAR-NK cells were incubated with either HL-60 or KG-1 cells for 4 hours. The supernatant was collected to assess for the IFN-γ level by ELISA. (8C) CD107a degranulation assays of single and dual CAR-NK cells cocultured with different target cells. CAR-NK cells were incubated with HL-60 or KG-1 for 1 hour at 37° C. with a PE-conjugated anti-CD107a antibody. Monensin (GolgiStop) was added to the cell culture. After incubation for 4 hours, cells were stained with an anti-CD56 antibody. The CD107a+ population in CD56+ cells was determined by flow cytometry. Data are shown as mean±SEM of two independent experiments. Statistical significance is calculated by unpaired two-tailed Student's t-test. **** p<0.0001; *** p<0.001; ns: not significant.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed, Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

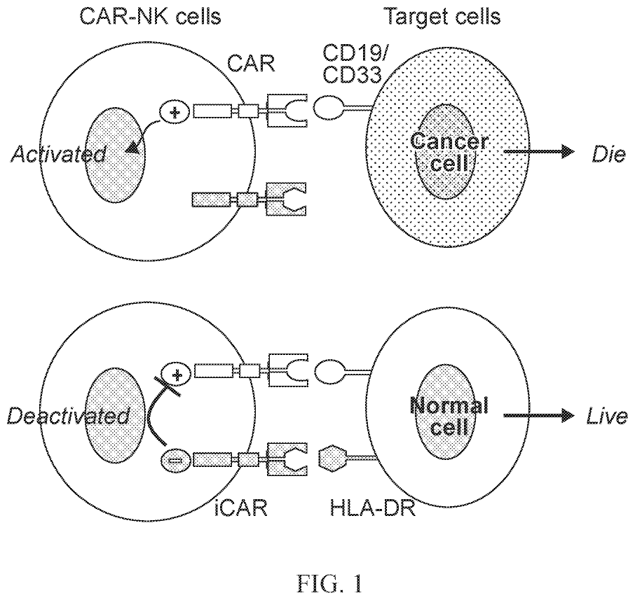

We conceive that the anti-cancer specificity of CAR-NK cells can be enhanced by activating them in response to cancer antigens while inhibiting them in response to HLA-DR (FIG. 1). An inhibitory CAR (iCAR), which contained an intracellular domain derived from immunosuppressive receptors PD-1 or CTLA-4, could inhibit T cell activation in response to the target antigen. Herein we investigated whether an iCAR could be designed to inhibit unwanted CAR-NK cell activation against HLA-DR+cells.

A PD-1-based anti-HLA-DR iCAR is provided that can effectively inhibit NK cells in response to HLA-DR expression on target cells. We show that dual CAR-NK cells, which co-express the anti-HLA-DR iCAR with a CD28/CD3ζ-based anti-CD19 CAR, can preferentially target CD19⁺HLA-DR^{neg} cells over CD19⁺HLA-DR⁺ cells. We additionally find that the iCAR-mediated inhibition is positively correlated with the densities of both the iCAR and HLA-DR. We also find that HLA-DR-expressing surrounding cells do not affect the target selectivity of dual CAR-NK cells. Furthermore, we confirm that HLA-DR-positive cells are resistant to dual CAR-NK cell-mediated killing in vivo using a xenograft mouse model. Finally, we show that the anti-HLA-DR iCAR is also compatible with the anti-CD33 CAR, enabling NK cells to preferentially target HLA-DR-negative AML cells. Our study lays a solid foundation for the future development of safer CAR-NK cell therapy against malignancies with HLA-DR loss.

An inhibitory CAR can have an extracellular domain recognizing a target antigen (e.g., a cancer-specific antigen) and an intracellular domain derived from immunosuppressive receptors PD-1 or CTLA-4. In some implementations, the inhibitory CAR includes the transmembrane domains and the amino acids up to the first annotated extracellular topological domain (for PD-1, amino acids 145 to 288; for CTLA-4, amino acids 161 to 223), so as to use the endogenous hinge region of each receptor; the intracellular tail of PD-1 or CTLA-4; and an antigen-specific targeting domain based on, for example, an antigen-specific single chain Fv (scFv) fragment.

In some embodiments, the antigen-specific targeting domain targets an antigen selected from the group consisting of antigens specific for cancer, an inflammatory disease, a neuronal disorder, diabetes, a cardiovascular disease, an infectious disease, an autoimmune disease, and combinations thereof.

Exemplary antigens specific for cancer include CD19, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD20, CD200, CD22, CD221, CD23 (IgE receptor), CD28, CD30 (TNFRSF8), CD33, CD123, CLL-1 (CLEC12A), MUC1, CD4, CD40, CD44 v6, CD51, CD52, CD56, CD74, CD80, CEA, CNTO888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin a5p1, integrin avP3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-P, TRAIL-R1, TRAIL-R2, tumor antigen CTAA16.88, VEGF-A, VEGFR-1, VEGFR2, vimentin, and combinations thereof. In various embodiments, the antigens specific for cancer include CD33, CD123, CLL-1 (CLEC12A), MUC1 or combinations thereof.

Exemplary antigens specific for an inflammatory disease include AOC3 (VAP-1), CAM-3001, CCL11 (eotaxin-1), CD125, CD147 (basigin), CD154 (CD40L), CD2, CD20, CD23 (IgE receptor), CD25 (α chain of IL-2 receptor), CD3, CD4, CD5, IFN-α, IFN-7, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, Lama glama, LFA-1 (CD11a), MEDI-528, myostatin, OX-40, rhuMAb β7, scleroscin, SOST, TGF beta 1, TNF-α, VEGF-A, and combinations thereof.

Exemplary antigens specific for a neuronal disorder include beta amyloid, MABT5102A, and combinations thereof.

Exemplary antigens specific for diabetes include L-10, CD3, and combinations thereof.

Exemplary antigens specific for a cardiovascular disease include C5, cardiac myosin, CD41 (integrin alpha-IIb), fibrin II, beta chain, ITGB2 (CD18), sphingosine-1-phosphate, and combinations thereof.

Exemplary antigens specific for an infectious disease include anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus, TNF-α, and combinations thereof.

In some implementations, the antigen-specific targeting domain of the inhibitory CAR is based on a human anti-HLA-DR antibody 1D09C3, or another anti-HLA-DR monoclonal antibody clone L243, available from Becton Dickinson (San Jose, CA). Clone L243 is derived from the hybridization of NS-1/1-Ag4 mouse myeloma cells with spleen cells isolated from BALB/c mice immunized with the human lymphoblastoid B-cell line RPMI 8866.

In some embodiments, the antigen-specific targeting domain of the inhibitory CAR is based on antibodies such as 305D3, 1C7277, or B8. For example, a full length heavy chain, variable region of the heavy chain (VH), an Fab fragment, a scFv of those antibodies.

An antigen-specific targeting domain can be in the form of a full length heavy chain, variable region of the heavy chain (VH), an Fab fragment, a scFv, a divalent single chain antibody or a diabody, each of which is specific to the target antigen.

Various embodiments of the present invention provide for a genetically engineered immune cell expressing a first chimeric antigen receptor (CAR) and a second, inhibitory CAR, by introduction of one or more genes, wherein the first CAR comprises: a first antigen-specific binding domain, a first transmembrane domain, and a first intracellular domain; and the second, inhibitory CAR comprises: a second antigen-specific binding domain, which is specific for and binds a human leukocyte antigen (HLA), a second transmembrane domain, and a second intracellular domain, which is a signaling domain or intracellular tail of an immunoinhibitory receptor.

In various embodiments, the genetically engineered immune cell is a natural killer (NK) cell. In various embodiments, the genetically engineered immune cell is a T cell. In various embodiments, the genetically engineered immune cell is more selective in targeting a malignant cell or tumor cell that expresses an antigen that the first antigen-specific binding domain binds to but does not express the HLA, than targeting a malignant cell or tumor cell that expresses both the antigen that the first antigen-specific binding domain binds to and the HLA.

In various embodiments, the second antigen-specific binding domain is specific for and binds HLA-DR, and the genetically engineered immune cell is an NK cell that does not express HLA-DR.

In various embodiments, the second antigen-specific binding domain comprises: a single-chain variable fragment (scFv) from clone 1D09C3 (e.g., SEQ ID NO:7). In various embodiments, the second antigen-specific binding domain comprises: a single-chain variable fragment (scFv) having the sequence as set forth in SEQ ID NO:7 or at least 90% identical to SEQ ID NO:7. In various embodiments, the scFv is at least 95% identical to SEQ ID NO:7. In various embodiments, the scFv is at least 99% identical to SEQ ID NO:7.

In various embodiments, the second antigen-specific binding domain comprises: a variable region of the light chain (VL) and a variable region of the heavy chain from clone 1D09C3 (e.g., SEQ ID NO:8 and 9, respectively). In various embodiments, the second antigen-specific binding domain comprises: a variable region of the light chain (VL) having the sequence as set forth in SEQ ID NO:8 or at least 90% identical to SEQ ID NO:8, and a variable region of the heavy chain having the sequence as set forth in SEQ ID NO:9 or at least 90% identical SEQ ID NO:9. In various embodiments, the VL is at least 95% identical to SEQ ID NO:8. In various embodiments, the VL is at least 99% identical to SEQ ID NO:8. In various embodiments, the VH is at least 95% identical to SEQ ID NO:9. In various embodiments, the VH is at least 95% identical to SEQ ID NO:9.

In various embodiments, the second antigen-specific binding domain further comprises a linker between the VL and the VH. In various embodiment the linker comprises (GGGGS)n (SEQ ID NO:10), (GGGS)n (SEQ ID NO:11), (SGGGG)n (SEQ ID NO:12) or (SGGG)n (SEQ ID NO:13) wherein n is an integer between 1 and 20. In various embodiments, n is an integer between 1 and 10. In various embodiments, n is an integer between 1 and 5. In various embodiments, n is an integer between 1 and 3. In various embodiments, linker comprises RGRGRGRGRSRGGGS (SEQ ID NO:14). Additional examples of linkers that can be used can be found in Lou and Cao, “Antibody variable region engineering for improving cancer immunotherapy,” Cancer Communications. 2022; 42:804-827, incorporated herein by reference as though fully set forth.

In various embodiments, second antigen-specific binding domain comprises: a single-chain variable fragment (scFv) from anti-HLA-DR monoclonal antibody clone L243. In various embodiments, second antigen-specific binding domain comprises: a variable region of the light chain (VL) and a variable region of the heavy chain (VH) from anti-HLA-DR monoclonal antibody clone L243. In various embodiments, the second antigen-specific binding domain further comprises a linker between the VL and the VH. The linker can be those as described herein.

In various embodiments, second antigen-specific binding domain comprises: a single-chain variable fragment (scFv) from anti-HLA-DR monoclonal antibody clone 305D3, 1C7277, or B8. In various embodiments, second antigen-specific binding domain comprises: a variable region of the light chain (VL) and a variable region of the heavy chain (VH) from anti-HLA-DR monoclonal antibody 305D3, 1C7277, or B8. In various embodiments, the second antigen-specific binding domain further comprises a linker between the VL and the VH. The linker can be those as described herein.

In various embodiments, second antigen-specific binding domain comprises: a single-chain variable fragment (scFv) from an anti-HLA-DR monoclonal antibody known in the art as of the filing date of the present application. In various embodiments, second antigen-specific binding domain comprises: a variable region of the light chain (VL) and a variable region of the heavy chain (VH) from anti-HLA-DR monoclonal antibody known in the art as of the filing date of the present application. In various embodiments, the second antigen-specific binding domain further comprises a linker between the VL and the VH. The linker can be those as described herein.

In various embodiments, the second antigen-specific binding domain comprises a single-chain variable fragment (scFv) from a monoclonal antibody known in the art as of the filing date of the present application, the known monoclonal antibody being an antibody that specifically binds to an antigen selected from the group consisting of CD19, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD20, CD200, CD22, CD221, CD23 (IgE receptor), CD28, CD30 (TNFRSF8), CD33, CD123, CLL-1 (CLEC12A), MUC1, CD4, CD40, CD44 v6, CD51, CD52, CD56, CD74, CD80, CEA, CNTO888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

In various embodiments, second antigen-specific binding domain comprises: a variable region of the light chain (VL) and a variable region of the heavy chain (VH) from a monoclonal antibody known in the art as of the filing date of the present application, the known monoclonal antibody being an antibody that specifically binds to an antigen selected from the group consisting of CD19, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD20, CD200, CD22, CD221, CD23 (IgE receptor), CD28, CD30 (TNFRSF8), CD33, CD123, CLL-1 (CLECi2A), MUC1, CD4, CD40, CD44 v6, CD51, CD52, CD56, CD74, CD80, CEA, CNTO888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

In various embodiments, the second antigen-specific binding domain comprises a single-chain variable fragment (scFv) from a monoclonal antibody known in the art as of the filing date of the present application, the known monoclonal antibody being an antibody that specifically binds to an antigen selected from the group consisting of AOC3 (VAP-1), CAM-3001, CCL11 (eotaxin-1), CD125, CD147 (basigin), CD154 (CD40L), CD2, CD20, CD23 (IgE receptor), CD25 (α chain of IL-2 receptor), CD3, CD4, CD5, IFN-α, IFN-7, IgE, IgE Fc region, IL-1, IL-12, IL-23, IL-13, IL-17, IL-17A, IL-22, IL-4, IL-5, IL-5, IL-6, IL-6 receptor, integrin α4, integrin α407, Lama glama, LFA-1 (CD11 a), MEDI-528, myostatin, OX-40, rhuMAb 07, scleroscin, SOST, TGF beta 1, TNF-α, VEGF-A, and combinations thereof.

In various embodiments, second antigen-specific binding domain comprises: a variable region of the light chain (VL) and a variable region of the heavy chain (VH) from a monoclonal antibody known in the art as of the filing date of the present application, the known monoclonal antibody being an antibody that specifically binds to an antigen selected from the group consisting of AOC3 (VAP-1), CAM-3001, CCL11 (eotaxin-1), CD125, CD147 (basigin), CD154 (CD40L), CD2, CD20, CD23 (IgE receptor), CD25 (α chain of IL-2 receptor), CD3, CD4, CD5, IFN-α, IFN-7, IgE, IgE Fc region, IL-1, IL-12, IL-23, IL-13, IL-17, IL-17A, IL-22, IL-4, IL-5, IL-5, IL-6, IL-6 receptor, integrin α4, integrin α407, Lama glama, LFA-1 (CD11a), MEDI-528, myostatin, OX-40, rhuMAb 07, scleroscin, SOST, TGF beta 1, TNF-α, VEGF-A, and combinations thereof.

In various embodiments, the second antigen-specific binding domain comprises a single-chain variable fragment (scFv) from a monoclonal antibody known in the art as of the filing date of the present application, the known monoclonal antibody being an antibody that specifically binds to an antigen selected from the group consisting of antigens specific for a neuronal disorder include beta amyloid, MABT5102A, and combinations thereof.

In various embodiments, second antigen-specific binding domain comprises: a variable region of the light chain (VL) and a variable region of the heavy chain (VH) from a monoclonal antibody known in the art as of the filing date of the present application, the known monoclonal antibody being an antibody that specifically binds to an antigen selected from the group consisting of antigens specific for a neuronal disorder include beta amyloid, MABT5102A, and combinations thereof.

In various embodiments, the second antigen-specific binding domain comprises a single-chain variable fragment (scFv) from a monoclonal antibody known in the art as of the filing date of the present application, the known monoclonal antibody being an antibody that specifically binds to an antigen selected from the group consisting of specific for diabetes include L-1β, CD3, and combinations thereof.

In various embodiments, second antigen-specific binding domain comprises: a variable region of the light chain (VL) and a variable region of the heavy chain (VH) from a monoclonal antibody known in the art as of the filing date of the present application, the known monoclonal antibody being an antibody that specifically binds to an antigen selected from the group consisting of specific for diabetes include L-1β, CD3, and combinations thereof.

In various embodiments, the second antigen-specific binding domain comprises a single-chain variable fragment (scFv) from a monoclonal antibody known in the art as of the filing date of the present application, the known monoclonal antibody being an antibody that specifically binds to an antigen selected from the group consisting of C5, cardiac myosin, CD41 (integrin alpha-IIb), fibrin II, beta chain, ITGB2 (CD18), sphingosine-1-phosphate, and combinations thereof.

In various embodiments, second antigen-specific binding domain comprises: a variable region of the light chain (VL) and a variable region of the heavy chain (VH) from a monoclonal antibody known in the art as of the filing date of the present application, the known monoclonal antibody being an antibody that specifically binds to an antigen selected from the group consisting of C5, cardiac myosin, CD41 (integrin alpha-IIb), fibrin II, beta chain, ITGB2 (CD18), sphingosine-1-phosphate, and combinations thereof.

In various embodiments, the second antigen-specific binding domain comprises a single-chain variable fragment (scFv) from a monoclonal antibody known in the art as of the filing date of the present application, the known monoclonal antibody being an antibody that specifically binds to an antigen selected from the group consisting of antigens from anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus, TNF-α, and combinations thereof.

In various embodiments, second antigen-specific binding domain comprises: a variable region of the light chain (VL) and a variable region of the heavy chain (VH) from a monoclonal antibody known in the art as of the filing date of the present application, the known monoclonal antibody being an antibody that specifically binds to an antigen selected from the group consisting of antigens from anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus, TNF-α, and combinations thereof.

In various embodiments, the second intracellular domain is the signaling domain or intracellular tail of an immunoinhibitory receptor selected from the group consisting of programmed cell death protein 1 (PD-1), cytotoxic T lymphocyte-associated antigen-4 (CTLA-4), lymphocyte-activation gene 3 (LAG-3), 2B4, B- and T-lymphocyte attenuator (BTLA), T cell immunoglobulin and ITIM domain (TIGIT), V-domain immunoglobulin suppressor of T cell activation (VISTA), NKG2A, and a combination thereof.

In various embodiments, the second intracellular domain is the signaling domain or intracellular tail of PD-1. In various embodiments, the second intracellular domain is CTLA-4.

In various embodiments, the first antigen-specific binding domain is specific for and binds an antigen selected from the group consisting of antigens specific for cancer, an inflammatory disease, a neuronal disorder, diabetes, a cardiovascular disease, an infectious disease, an autoimmune disease, and a combination thereof.

In various embodiments, the first antigen-specific binding domain is specific for and binds CD33.

In various embodiments, the first antigen-specific binding domain comprises: a single-chain variable fragment (scFv) from clone my96 (e.g., SEQ ID NO:4).

In various embodiments, the first antigen-specific binding domain comprises: a single-chain variable fragment (scFv) having the sequence as set forth in SEQ ID NO:4 or at least 90% identical to SEQ ID NO:4. In various embodiments, the scFv has a sequence at least 95% identical to SEQ ID NO:4. In various embodiments, the scFv has a sequence at least 96%, 97%, 98% or 99% identical to SEQ ID NO:4.

In various embodiments, the first antigen-specific binding domain comprises: a variable region of the light chain (VL) and a variable region of the heavy chain from clone my96 (e.g., SEQ ID NO:5 and 6, respectively).

In various embodiments, the first antigen-specific binding domain comprises: a variable region of the light chain (VL) having the sequence as set forth in SEQ ID NO:5 or at least 90% identical to SEQ ID NO:5, and a variable region of the heavy chain (VH) having the sequence as set forth in SEQ ID NO:6 or at least 90% identical SEQ ID NO:6. In various embodiments the VL has a sequence at least 95% identical to SEQ ID NO:5. In various embodiments the VL has a sequence at least 96%, 97%, 98% or 99% identical to SEQ ID NO:5. In various embodiments the VH has a sequence at least 95% identical to SEQ ID NO:6. In various embodiments the VH has a sequence at least 96%, 97%, 98% or 99% identical to SEQ ID NO:6. In various embodiments, the first antigen-specific binding domain further comprises a linker between the VL and the VH.

In various embodiments, the second antigen-specific binding domain further comprises a linker between the VL and the VH. In various embodiment the linker comprises (GGGGS)n (SEQ ID NO:10), (GGGS)n (SEQ ID NO:11), (SGGGG)n (SEQ ID NO:12) or (SGGG)n (SEQ ID NO:13) wherein n is an integer between 1 and 20. In various embodiments, n is an integer between 1 and 10. In various embodiments, n is an integer between 1 and 5. In various embodiments, n is an integer between 1 and 3. In various embodiments, linker comprises RGRGRGRGRSRGGGS (SEQ ID NO:14). Additional examples of linkers that can be used can be found in Lou and Cao, “Antibody variable region engineering for improving cancer immunotherapy,” Cancer Communications. 2022; 42:804-827, incorporated herein by reference as though fully set forth.

In various embodiments, first antigen-specific binding domain comprises: a single-chain variable fragment (scFv) from a monoclonal antibody my96. In various embodiments, first antigen-specific binding domain comprises: a variable region of the light chain (VL) and a variable region of the heavy chain (VH) from a monoclonal antibody monoclonal antibody my96. In various embodiments, the second antigen-specific binding domain further comprises a linker between the VL and the VH. The linker can be those as described herein.

In various embodiments, first antigen-specific binding domain comprises: a single-chain variable fragment (scFv) from a monoclonal antibody specific for and binds CD33 known in the art as of the filing date of the present application. In various embodiments, first antigen-specific binding domain comprises: a variable region of the light chain (VL) and a variable region of the variable region of the heavy chain (VH) from a monoclonal antibody specific for and binds CD33 known in the art as of the filing date of the present application. In various embodiments, the second antigen-specific binding domain further comprises a linker between the VL and the VH. The linker can be those as described herein.

In various embodiments, the first antigen-specific binding domain is specific for and binds CD19.

In various embodiments, the first antigen-specific binding domain comprises: a single-chain variable fragment (scFv) from clone FMC63 (e.g., SEQ ID NO:1). In various embodiments, the first antigen-specific binding domain comprises: a single-chain variable fragment (scFv) having the sequence as set forth in SEQ ID NO:1 or at least 90% identical to SEQ ID NO:1. In various embodiments, the first antigen-specific binding domain comprises: scFv having a sequence at least 95% identical to SEQ ID NO:1. In various embodiments, the first antigen-specific binding domain comprises: scFv having a sequence at least 96%, 97%, 98% or 99% identical to SEQ ID NO:1.

In various embodiments, the first antigen-specific binding domain comprises: a variable region of the light chain (VL) and a variable region of the heavy chain from clone FMC63 (e.g., SEQ ID NO:2 and 3, respectively). In various embodiments, the first antigen-specific binding domain comprises: a variable region of the light chain (VL) having the sequence as set forth in SEQ ID NO:2 or at least 90% identical to SEQ ID NO:2, and a variable region of the heavy chain (VH) having the sequence as set forth in SEQ ID NO:3 or at least 90% identical SEQ ID NO:3.

In various embodiments, the first antigen-specific binding domain comprises: a VL having a sequence at least 95% identical to SEQ ID NO:2, and VH having a sequence at least 95% identical SEQ ID NO:3. In various embodiments, the first antigen-specific binding domain comprises: a VL having a sequence at least 96%, 97%, 98%, or 99% identical to SEQ ID NO:2, and VH having a sequence at least 96%, 97%, 98%, or 99% identical SEQ ID NO:3.

In various embodiments, the second antigen-specific binding domain further comprises a linker between the VL and the VH. In various embodiment the linker comprises (GGGGS)n (SEQ ID NO:10), (GGGS)n (SEQ ID NO:11), (SGGGG)n (SEQ ID NO:12) or (SGGG)n (SEQ ID NO:13) wherein n is an integer between 1 and 20. In various embodiments, n is an integer between 1 and 10. In various embodiments, n is an integer between 1 and 5. In various embodiments, n is an integer between 1 and 3. In various embodiments, linker comprises RGRGRGRGRSRGGGS (SEQ ID NO:14). Additional examples of linkers that can be used can be found in Lou and Cao, “Antibody variable region engineering for improving cancer immunotherapy,” Cancer Communications. 2022; 42:804-827, incorporated herein by reference as though fully set forth.

In various embodiments, first antigen-specific binding domain comprises: a single-chain variable fragment (scFv) from a monoclonal antibody specific for and binds CD19 known in the art as of the filing date of the present application. In various embodiments, first antigen-specific binding domain comprises: a variable region of the light chain (VL) and a variable region of the heavy chain (VH) from a monoclonal antibody specific for and binds CD19 known in the art as of the filing date of the present application. In various embodiments, the second antigen-specific binding domain further comprises a linker between the VL and the VH. The linker can be those as described herein.

In various embodiments of the genetically engineered immune cell, the first transmembrane domain comprises CD28 transmembrane domain, CD8 transmembrane domain, a transmembrane domain of a zeta chain of a T cell receptor complex, or a combination thereof.

In various embodiments of the genetically engineered immune cell, the second transmembrane domain comprises a transmembrane domain of the immunoinhibitory receptor, CD28 transmembrane domain, CD8 transmembrane domain, a transmembrane domain of a zeta chain of a T cell receptor complex, or a combination thereof.

In various embodiments of the genetically engineered immune cell, the first CAR further comprises a first extracellular spacer domain, positioned between the first antigen-specific binding domain and the first transmembrane domain, and the first extracellular spacer domain comprises (i) a hinge region of CD8α, (ii) a hinge region of IgG4, (iii) a hinge and CH2 region of IgG4, (iv) a hinge, CH2 and CH3 region of IgG4, (v) a hinge, CH2 and CH3 region of IgG1, (vi) a hinge region of IgG1, (vi) a hinge and CH2 region of IgG1, or (vii) a combination of any two or more of (i)-(vi).

In various embodiments, the first extracellular spacer domain comprises IgG4 hinge-CH2-CH3.

In various embodiments, the first extracellular spacer domain comprises a hinge region of IgG1, IgG2, IgG3 or IgG4. In various embodiments, the first extracellular spacer domain comprises CH1 region, CH2 region or CH3 region of IgG1, IgG2, IgG3 or IgG4.

In various embodiments of the genetically engineered immune cell, the second, inhibitory CAR further comprises a second extracellular spacer domain, positioned between the second antigen-specific binding domain and the second transmembrane domain, and the second extracellular spacer domain comprises (1) an extracellular topological domain of the immunoinhibitory receptor, (2) a hinge region of CD8α, (3) a hinge region of IgG4, (4) a hinge and CH2 region of IgG4, (5) a hinge, CH2 and CH3 region of IgG4, (6) a hinge, CH2 and CH3 region of IgG1, (7) a hinge region of IgG1, (8) a hinge and CH2 region of IgG1, or (9) a combination of any two or more of (1)-(8).

In various embodiments, the second extracellular spacer domain comprises a hinge region of IgG4PD-1, CTLA-4, Tim-3, LAG-3, VISTA, TIGIT, or NKG2A. In various embodiments, the first extracellular spacer domain comprises CH1 region, CH2 region or CH3 region of IgG1, IgG2, IgG3 or IgG4.

In various embodiments of the genetically engineered immune cell, the first intracellular domain comprises one or more of a CD3 zeta chain, FcγRIII, FcεRI, a cytoplasmic tail of a Fc receptor, and an immunoreceptor tyrosine-based activation motif (ITAM) bearing cytoplasmic receptors; and optionally further comprising a co-stimulatory domain selected from the group consisting of CD28, CD137 (4-1BB), CD134 (OX40), Dap10, CD27, CD2, CD5, ICAM-1, LFA-1, Lck, TNFR-I, TNFR-II, Fas, CD30, CD40, NKG2D, 2B4 and a combination thereof.

In various embodiments of the genetically engineered immune cell, the first CAR, the second, inhibitory CAR, or both, further comprises a protein tag at N terminus; and the protein tag of the first CAR and that of the second, inhibitory CAR are different, if both are present.

Examples of protein tags include but are not limited to HA-tag, FLAG-tag, Myc-tag, V5-tag, and His6-tag.

In various embodiments of the genetically engineered immune cell, the secretion level of IFN-γ, after contact of the genetically engineered immune cell with a target cell expressing an antigen that the first antigen-specific binding domain is specific for and binds to is statistically similar to that of a genetically engineered immune cell that is otherwise identical except for lacking the second, inhibitory CAR.

In various embodiments of the genetically engineered immune cell, the cytotoxicity level towards the target cell expressing the antigen that the first antigen-specific binding domain is specific for and binds to, is statistically similar to that of a genetically engineered immune cell that is otherwise identical except for lacking the second, inhibitory CAR.

In various embodiments of the genetically engineered immune cell, the secretion level of IFN-γ after the contact with the target cell, whose degranulation if the genetically engineered immune cell is an NK cell upon incubation with a second target cell expressing the HLA, and/or whose cytotoxicity level towards the second target cell expressing the HLA, is at least 80%, 70%, 60%, 50%, 40%, 30%, or 20% lower, or at least two-fold lower, at least three-fold lower, at least five-fold lower, or at least ten-fold lower than the genetically engineered immune cell that is otherwise identical except for lacking the second, inhibitory CAR.

In various embodiments of the genetically engineered immune cell, the second, inhibitory CAR is encoded by a coding sequence in a gene construct introduced to the genetically engineered immune cell, and the second, inhibitory CAR is operably linked to a transcriptional promoter comprising UBC1, EF1a or MSCV.

Various embodiments of the present invention provide for a combination, comprising: a first genetically engineered immune cell expressing a first chimeric antigen receptor (CAR), wherein the first CAR comprises: a first antigen-specific binding domain, a first transmembrane domain, and a first intracellular domain; and a second genetically engineered immune cell expressing a second, inhibitory CAR, wherein the second, inhibitory CAR comprises: a second antigen-specific binding domain, which is specific for and binds a human leukocyte antigen (HLA), a second transmembrane domain, and a second intracellular domain, which is a signaling domain or intracellular tail of an immunoinhibitory receptor.

Various embodiments of the present invention provide for a genetically engineered natural killer (NK) cell expressing an inhibitory chimeric antigen receptor (CAR) by introduction of a gene, wherein the inhibitory CAR comprises: an antigen-specific binding domain, which is specific for and binds human leukocyte antigen DR (HLA-DR), a transmembrane domain, and an intracellular domain, which is a signaling domain or intracellular tail of an immunoinhibitory receptor of PD-1.

Various embodiments provide for a vector encoding the second, inhibitory CAR of the present invention as described herein.

Various embodiments provide for a vector encoding the first CAR of the present invention as described herein.

Various embodiments provide for a method of preparing a genetically engineered immune cell of the present invention as described herein, comprising introducing to an immune cell a first vector encoding the first CAR and a second vector encoding the second, inhibitory CAR.

In various embodiments, the first vector and the second vector are introduced sequentially.

In various embodiments, the method further comprises selecting the immune cell expressing the first CAR after the first vector is introduced to the immune cell, wherein the second vector is introduced to the selected immune cell expressing the first CAR, so as to produce the genetically engineered immune cell expressing both the first CAR and the second, inhibitory CAR.

Various embodiments provide for a method of treating a subject with a hematologic malignancy or a solid tumor, comprising: administering to the subject a pharmaceutical composition comprising an effective quantity of genetically engineered immune cells of the present invention as described herein.

In various embodiments, the subject is detected or diagnosed with complete or partial loss of HLA-DR in the hematologic malignancy. In various embodiments, the subject is detected or diagnosed with complete or partial loss of HLA-A, HLA-B and/or HLA-C in the solid tumor.

In various embodiments, subject has a hematologic malignancy. Examples of hematologic malignancy include but are not limited to acute myeloid leukemia (AML), classical Hodgkin lymphoma (CHL), chronic myelomonocytic leukemia (CMML), chronic myeloid leukemia (CML), or a combination thereof. In various embodiments, the subject is an AML patient with post-transplant relapse.

In various embodiments, the pharmaceutical composition is administered to bloodstream or bone marrow of the subject.

In various embodiments, the pharmaceutical compositions according to the invention may be formulated for delivery via any route of administration. “Route of administration” may refer to any administration pathway known in the art, including but not limited to parenteral, ocular, nasal, transmucosal, or intratumoral.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly.

In various embodiments, the genetically engineered immune cells is produced by introducing the one or more genes encoding the first CAR and the second, inhibitory CAR into allogeneic NK cells obtained from another subject.

In various embodiments, the method further comprises administering to the subject a T-cell receptor-based immunotherapy or donor lymphocyte infusion, so as to target cancer cells that express the HLA.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Generation and Characterization of Single and Dual CAR-NK Cells

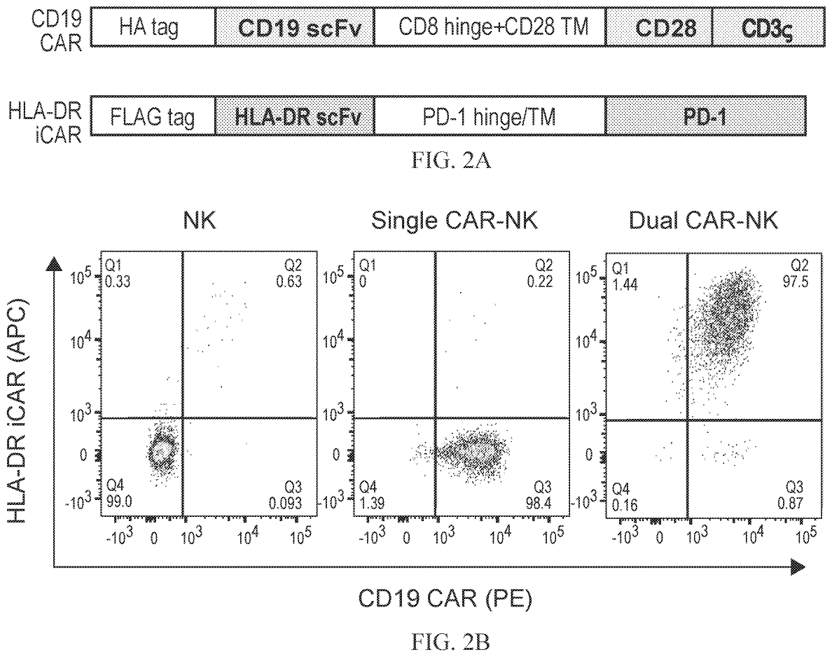

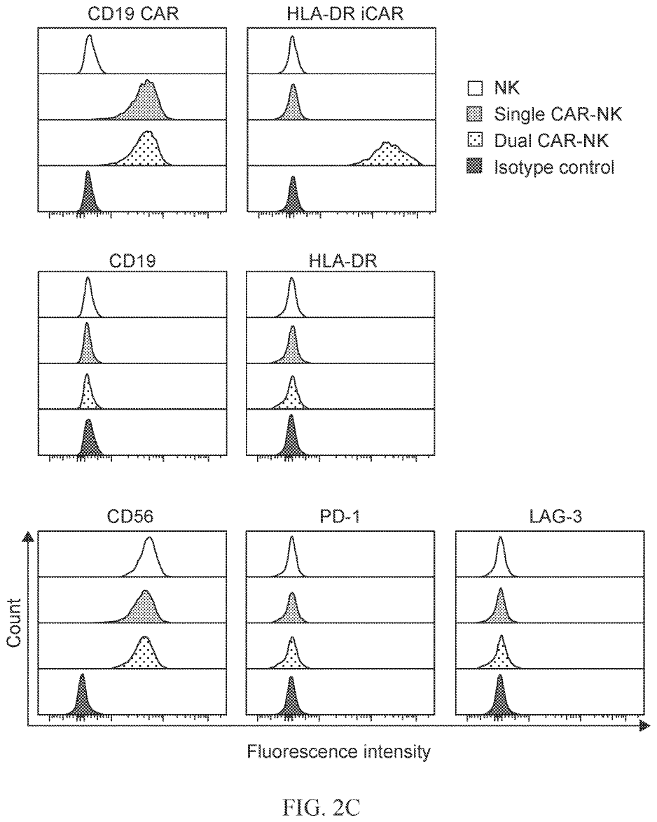

We first constructed an anti-HLA-DR iCAR to pair with a conventional anti-CD19 CAR (FIG. 2A). The anti-CD19 CAR consists of an N-terminal HA tag (for detecting CAR expression), a single-chain variable fragment (scFv) derived from the anti-CD19 antibody clone FMC63, a CD8a hinge domain, a CD28 transmembrane domain, and CD28/CD3C intracellular domains. The anti-HLA-DR iCAR consists of an N-terminal FLAG tag (for detecting iCAR expression), an extracellular scFv derived from the humanized anti-HLA-DR antibody 1D09C3 and PD-1 hinge, transmembrane, and intracellular domains. The CAR and iCAR DNAs were cloned into the pFUW lentiviral vector, and lentiviral particles were generated using HEK293T cells.

Next, we engineered NK-92MI cells-a human NK cell line-to express the anti-CD19 CAR with or without the anti-HLA-DR iCAR by lentiviral transduction. NK-92MI is an IL-2-expressing derivative of NK-92, both of which have been broadly used in the development of CAR-NK and other cellular therapeutics. In our study, NK-92MI cells were first transduced to generate anti-CD19 CAR-NK cells, hereafter referred to as “single CAR-NK cells”. After enrichment by fluorescence-activated cell sorting (FACS), single CAR-NK cells were transduced again to express the anti-HLA-DR iCAR. The cells expressing both CAR and iCAR were purified by FACS, referred to as “dual CAR-NK cells”. Flow cytometric analysis confirmed that both single and dual CAR-NK cells had a purity greater than 97% (FIG. 2B).

To ensure that the single and dual CAR-NK cells generated above were directly comparable, we further characterized their expression levels of CARs, antigens, and phenotype markers by surface staining and flow cytometry. Our observations were as follows: 1) single and dual CAR-NK cells expressed the anti-CD19 CAR at the same level, indicating that they would have a similar activation potential against CD19; 2) neither of them expressed CD19 or HLA-DR, indicating that they would not undergo anti-CD19 CAR-mediated fratricide or anti-HLA-DR iCAR-mediated self-inhibition; and 3) they had the same expression levels of the activation marker CD56 and the inhibitory receptors PD-1 and LAG-3 as unmodified NK-92MI cells, thus confirming a lack of phenotypic changes induced by CAR or iCAR expression (FIG. 2C). These results showed that single and dual CAR-NK cells were essentially identical except for the expression of the anti-HLA-DR iCAR.

### Verification of CD19 and HLA-DR Expression on Different Target Cells

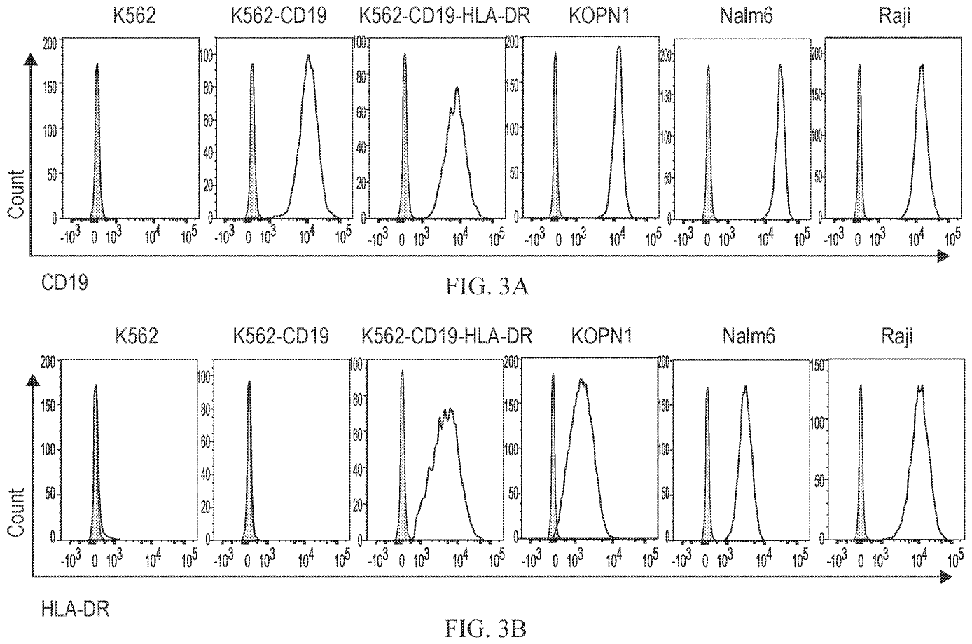

To examine the target specificity of single and dual CAR-NK cells, we selected a panel of six cell lines as target cells. These include K562 and its two derivatives: K562 is a CML cell line that is negative for both CD19 and HLA-DR; K562-CD19 is a modified cell line expressing CD19 but not HLA-DR; K562-CD19-HLA-DR is a new cell line we generated that expresses both CD19 and HLA-DR. The other three cell lines are KOPN1, Nalm6, and Raji, which are positive for CD19 and HLA-DR at different densities. We verified the expression (or lack thereof) of CD19 and HLA-DR on these cells by staining with PE-conjugated antibodies followed by flow cytometry (FIG. 3).

### Dual CAR-NK Cells Exhibit Reduced IFN-γ Production, CD69 Expression, Degranulation, and Cytotoxicity Against HLA-DR-Positive Cells In Vitro

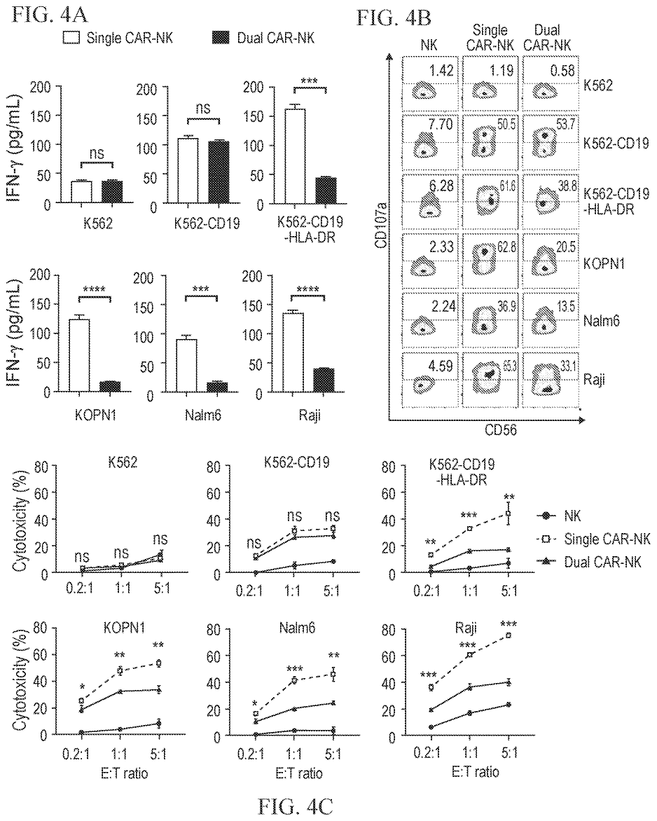

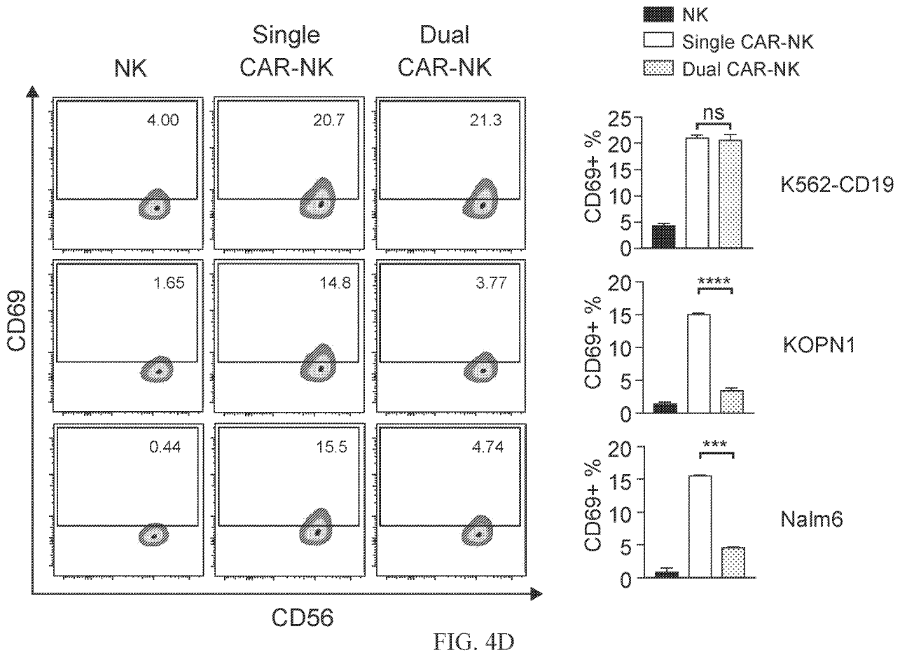

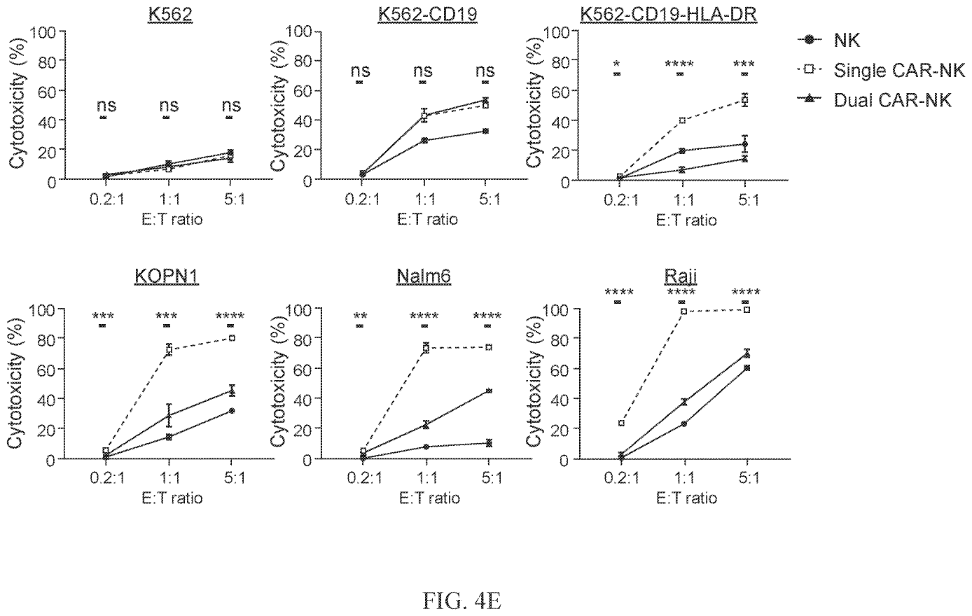

Next, we compared and contrasted the activation levels of single and dual CAR-NK cells against different target cells. We first assayed the production of interferon-y (IFN-γ), a cytokine indicator of NK cell activation and cytotoxicity. To this end, single and dual CAR-NK cells were incubated with each of the six target cells at a 1:1 effector-to-target (E:T) ratio for 4 hours, and the concentration of IFN-γ in the cell culture supernatant was measured using an enzyme-linked immunosorbent assay (ELISA). We found that both single and dual CAR-NK cells had enhanced IFN-γ production after incubation with K562-CD19 cells (CD19⁺HLA-DR^{neg}). Importantly, there was no significant difference between the amounts of IFN-γ they produced. Against K562-CD19-HLA-DR cells, however, dual CAR-NK cells had an approximately 70% reduction in IFN-γ production compared to single CAR-NK cells. A similar reduction was observed when dual CAR-NK cells targeted other HLA-DR-positive cells, including KOPN1, Nalm6, and Raji (FIG. 4A). These results show that dual CAR-NK cells were inhibited by target cells expressing HLA-DR.

We also monitored CD69 upregulation, an early marker of NK cell activation. We observed that single and dual CAR-NK cells had similar percentages of CD69⁺ population after incubation with K562-CD19 cells (20.7% and 21.3%, respectively). However, dual CAR-NK cells had a significantly lower CD69⁺ population than single CAR-NK cells after incubation with KOPN1 cells (3.77% versus 14.8%) or Nalm6 cells (4.74% versus 15.5%) (FIG. 4D). The results were thus consistent with those of the IFN-γ production assay.

To further assess NK cell activation, we performed a CD107a degranulation assay. For this purpose, effector cells (NK cells, single CAR-NK cells, and dual CAR-NK cells) were incubated with different target cells at a 1:1 E:T ratio for 4 hours. Cells were then stained for CD56 (a marker for NK cells) and CD107a (a marker for degranulation) and subjected to flow cytometric analysis. We demonstrated the following: 1) against K562 cells (negative control), neither single nor dual CAR-NK cells developed a CD107a+population, indicating no or little degranulation; 2) against K562-CD19 cells, both single and dual CAR-NK cells developed a CD107a+population (50.5% and 53.7%, respectively), indicating a similar level of degranulation; 3) against any of the four HLA-DR-positive target cells (K562-CD19-HLA-DR, KOPN1, Nalm6, and Raji), single CAR-NK cells consistently had a 2-3 fold higher percentage of CD107a+population than dual CAR-NK cells had, indicating that the latter were inhibited by HLA-DR-expressing targets (FIG. 4B).

Finally, we examined the cytotoxicity of single and dual CAR-NK cells against HLA-DR-positive and negative target cells. NK, single CAR-NK, and dual CAR-NK cells were co-cultured with different target cells at three E:T ratios (0.2:1, 1:1, 5:1). After a 4-hour incubation, the cytotoxicity was determined using a lactate dehydrogenase (LDH) release assay. The results showed that single and dual CAR-NK cells had similar killing ability against K562-CD19 cells. However, dual CAR-NK cells were significantly less cytotoxic than single CAR-NK cells in targeting HLA-DR⁺ cells, including K562-CD19-HLA-DR, KOPN1, Nalm6, and Raji (FIG. 4C). We additionally performed a flow cytometry-based cytotoxicity assay by determining the percentage of viable target cells after coculture. The results were consistent with the above, confirming that HLA-DR⁺ target cells were resistant to dual CAR-NK cell-mediated cytotoxicity (FIG. 4E).

Collectively, our data strongly supported that dual CAR-NK cells preferentially recognize and kill HLA-DR-negative cells over HLA-DR-positive cells.

The Inhibition of Dual CAR-NK Cells is Positively Correlated with Both HLA-DR and iCAR Densities

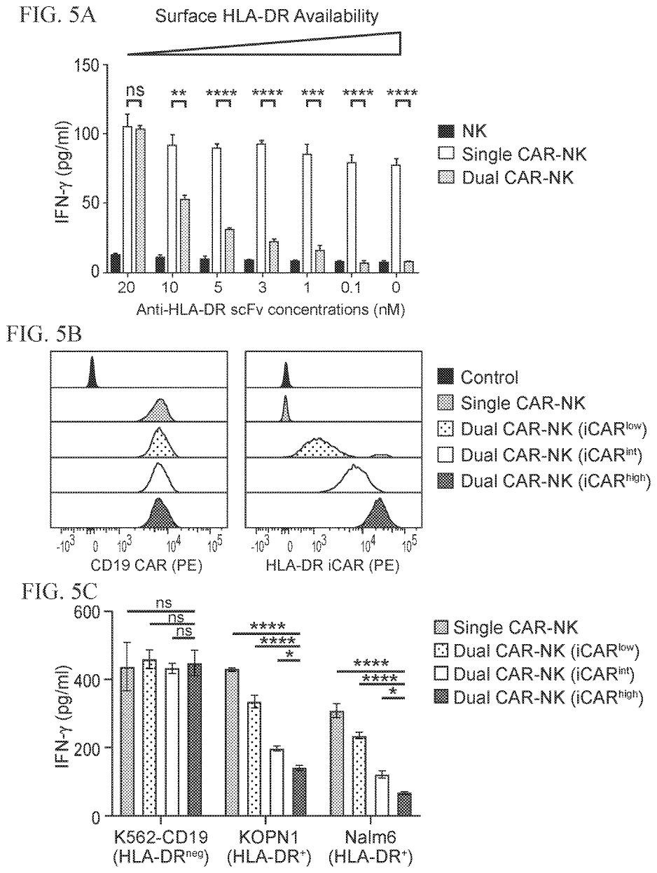

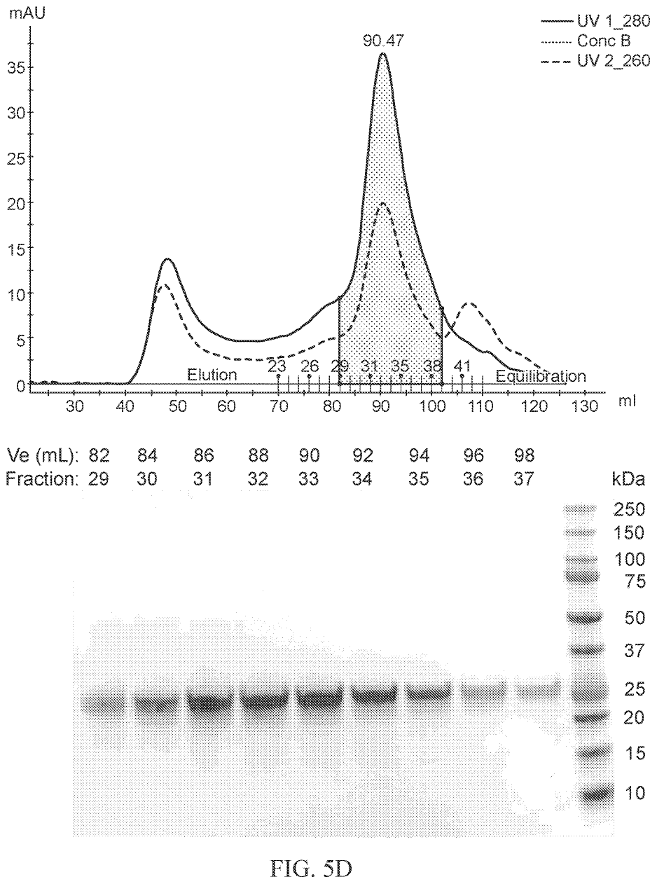

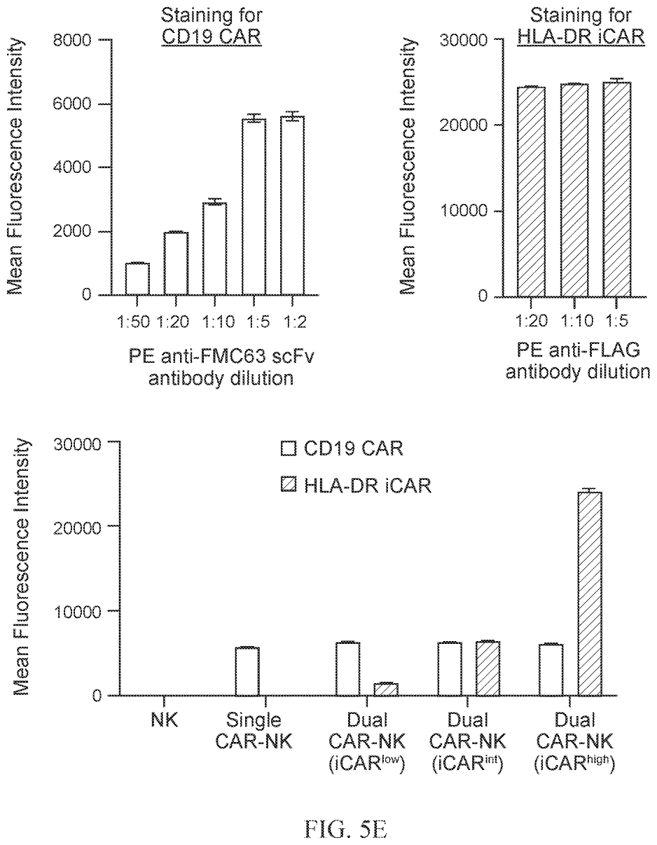

Our results showed that dual CAR-NK cells were inhibited by HLA-DR-positive cells but not by HLA-DR-negative cells. We hypothesized that the level of inhibition would be positively correlated with the densities of both HLA-DR on target cells and iCAR on NK cells. To test the effect of HLA-DR, we generated recombinant scFv (the same clone as the anti-HLA-DR iCAR) in E. coli followed by in vitro refolding and size-exclusion chromatography (FIG. 5D). We then used this scFv at different concentrations (0-20 nM) to block HLA-DR on the surface of K562-CD19-HLA-DR cells. This approach ensured that the only changing variable of target cells was the level of exposed HLA-DR, which was not attainable by directly comparing different cell lines as they could differ in other aspects. Next, we incubated single and dual CAR-NK cells with these target cells for 4 hours and assessed the activation of CAR-NK cells by IFN-γ production. We found that the activation level of dual CAR-NK cells gradually increased with increasing anti-HLA-DR scFv concentrations and reached that of single CAR-NK cells when scFv was used at 20 nM, the highest concentration tested (FIG. 5A). These results indicate that the anti-HLA-DR iCAR-mediated inhibition is positively correlated with the density of HLA-DR on the target cell surface.

To assess the effect of the iCAR density, we sorted dual CAR-NK cells into three populations that expressed iCAR at high, intermediate, and low densities, respectively. We verified them by staining with two separate antibodies: i) a PE-conjugated anti-FMC63 scFv antibody that recognizes the anti-CD19 CAR, and ii) a PE-conjugated anti-FLAG antibody that recognizes the anti-HLA-DR iCAR via its N-terminal FLAG tag. Flow cytometric analysis confirmed that they expressed the iCAR at varying levels, while the CAR was the same level as on single CAR-NK cells (FIG. 5B). Since both antibodies were used at saturating concentrations (FIG. 5E), it was reasonable to assume that the mean fluorescence intensities of stained cells were directly proportional to the level of CAR or iCAR. Based on this, we estimated that the iCAR-to-CAR ratios were 0.25:1, 1:1, and 4:1 for the iCAR^{low}, iCAR^{int}, and iCAR^{high} populations, respectively (FIG. 5E). Next, we compared the activation levels of these three dual CAR-NK cells as well as the single CAR-NK cells against different target cells. IFN-γ secretion assays showed that they performed equally well against HLA-DR-negative target cells. However, against HLA-DR-negative target cells, the activation level decreased in the following order: single CAR-NK (i.e., iCAR^{neg})>dual CAR-NK (iCAR^{low})>dual CAR-NK (iCAR^{int})>dual CAR-NK (iCAR^{high}) (FIG. 5C). These results show that the anti-HLA-DR iCAR-mediated inhibition is positively correlated with the level of iCAR (or the iCAR/CAR ratios) on NK cells.

### HLA-DR⁺ Surrounding Cells do not Affect the Target Selectivity of Dual CAR-NK Cells

We showed that dual CAR-NK cells were active towards CD19⁺HLA-DR^{neg} cells but inhibited by CD19⁺HLA-DR⁺ cells in vitro. A key question was whether the target selectivity of dual CAR-NK cells would be affected by HLA-DR-positive cells in proximity to the CD19⁺HLA-DR^{neg} target cells (e.g., in the bloodstream or bone marrow microenvironment).

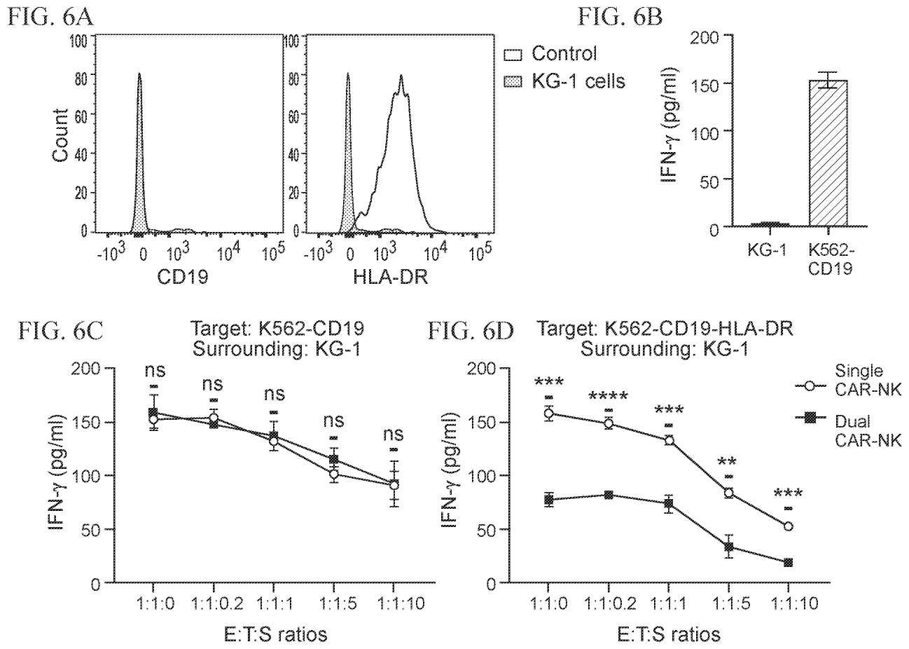

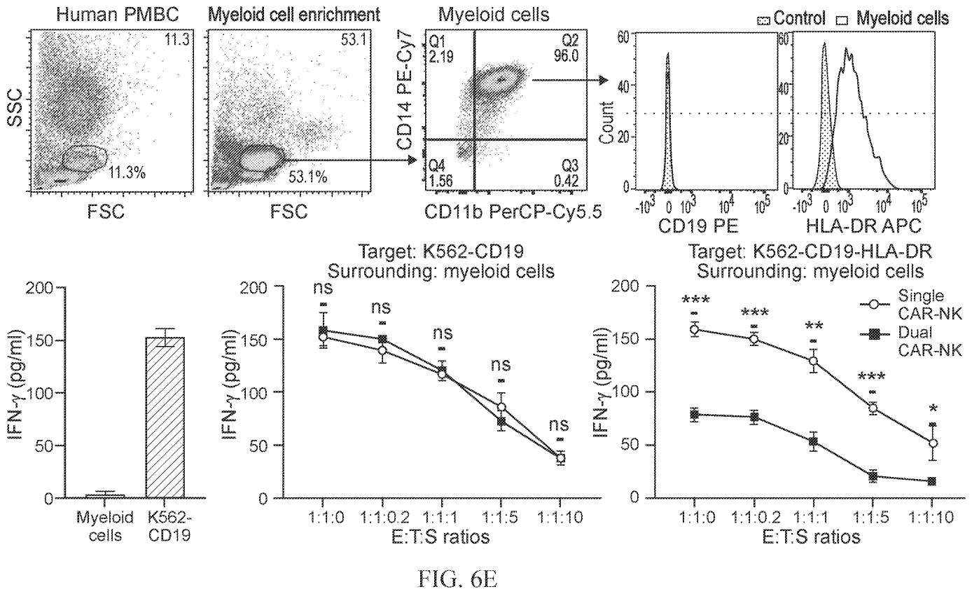

To address this concern, we used the KG-1 cell line as a model of surrounding cells. KG-1 cells expressed HLA-DR but not CD19, as shown by flow cytometry (FIG. 6A), and they alone could not activate anti-CD19 CAR-NK cells, as confirmed by cell coculture and IFN-γ production analysis (FIG. 6B). We then compared the activation levels of single and dual CAR-NK cells against K562-CD19 cells (CD19⁺HLA-DR^{neg}) in the presence or absence of KG-1 cells at five different levels, ranging from none to 10 times of target cells. With increasing KG-1 cells, we found that both single and dual CAR-NK cells gradually reduced IFN-γ production, probably because they had a decreasing chance of finding K562-CD19 cells in the mixture. Nevertheless, dual CAR-NK cells remained equally reactive as single CAR-NK cells under all conditions, indicating that they were not inhibited by HLA-DR⁺ surrounding cells (FIG. 6C). Next, we performed the same experiment using K562-CD19-HLA-DR cells (CD19⁺HLA-DR⁺) as target cells. We found that dual CAR-NK cells were consistently and considerably less reactive than single CAR-NK cells, indicating that the iCAR-mediated protective effect was not compromised by surrounding KG-1 cells (FIG. 6D).

To validate the above findings, we additionally used primary human myeloid cells as surrounding cells. For this purpose, we used immunomagnetic negative selection to isolate human monocytes (CD11b⁺CD14⁺), the main type of HLA-DR⁺ myeloid cells, from human peripheral blood mononuclear cells (PBMC) (FIG. 6E). Like the KG-1 cell line, these primary cells expressed HLA-DR but did not express CD19 or stimulate anti-CD19 CAR-NK cells (FIG. 6E). Next, we used them to substitute for KG-1 cells in the CAR-NK cell activation assays described above. The results again showed that dual CAR-NK cells were equally effective as single CAR-NK cells in targeting CD19⁺HLA-DR^{neg} cells but significantly less reactive than the latter in targeting CD19⁺HLA-DR⁺ cells (FIG. 6E).

Collectively, these results show that the target selectivity of dual CAR-NK cells is not affected by HLA-DR-positive surrounding cells.

### HLA-DR-Positive Cells are Resistant to Dual CAR-NK Cell-Mediated Killing In Vivo

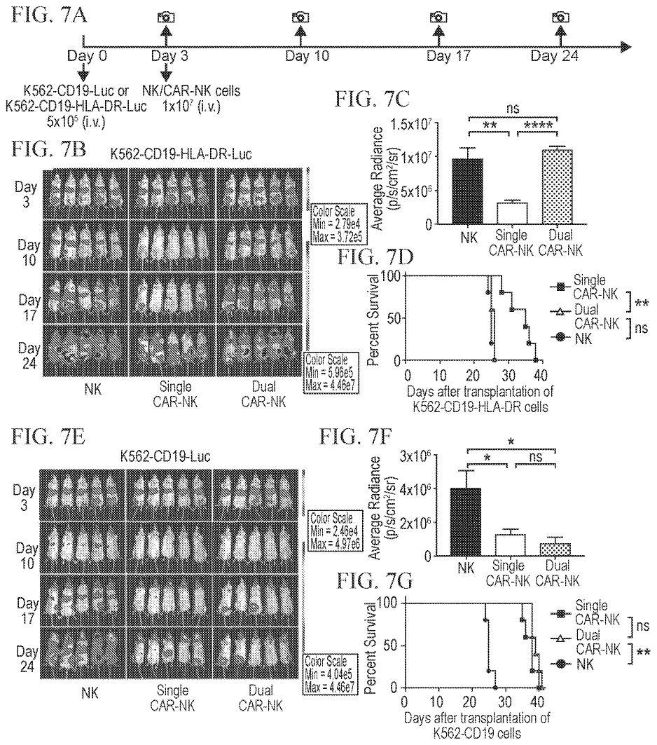

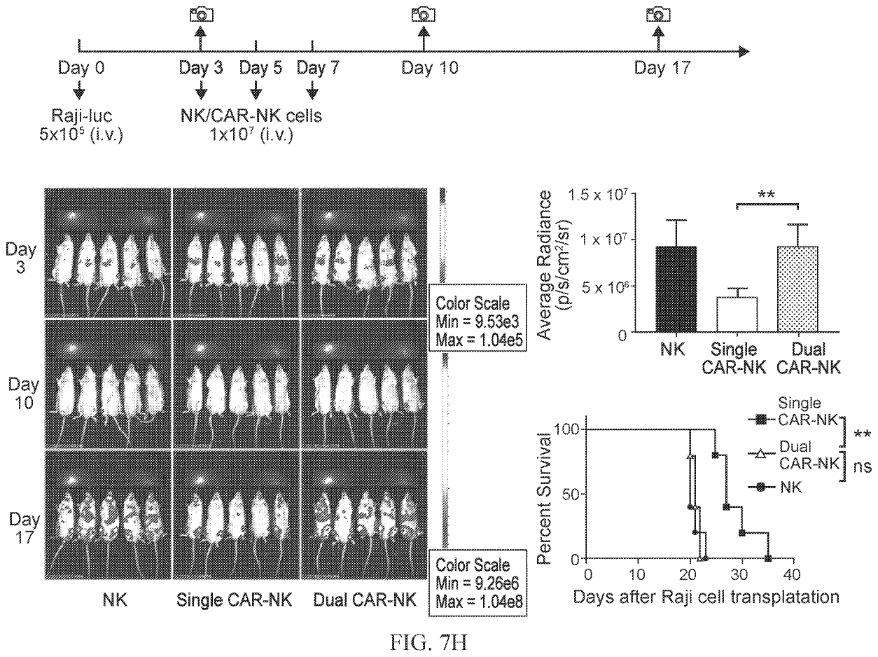

To further evaluate HLA-DR-mediated inhibition of dual CAR-NK cells, we performed an in vivo killing assay using mouse xenograft models. We first compared the ability of single and dual CAR-NK cells to kill K562-CD19-HLA-DR cells (CD19⁺HLA-DR⁺, as a model of HLA-DR-expressing cells). Immunocompromised NOD/SCID/gamma (NSG) mice were inoculated with firefly luciferase-expressing K562-CD19-HLA-DR cells. Mice were then treated with either unmodified NK cells (control), single CAR-NK cells, or dual CAR-NK cells (FIG. 7A). The growth of K562-CD19-HLA-DR cells in mice was monitored by bioluminescence imaging. On day 3, there was no visible difference among all three groups. On days 10, 17, and 24, we observed a much weaker bioluminescence signal in mice treated with single CAR-NK cells than in those treated with dual CAR-NK cells or NK cells (FIGS. 7B and 7C). We continued to monitor the survival of mice and found that the group treated with dual CAR-NK cells had a shorter survival time than those treated with single CAR-NK cells (FIG. 7D). Similar results were observed in a separate experiment in which we used Raji cells (CD19⁺HLA-DR⁺) as target cells (FIG. 7H). Thus, it is clear that dual CAR-NK cells have diminished cytotoxicity in vivo against target cells expressing both CD19 and HLA-DR.

We additionally compared the in vivo killing ability of single and dual CAR-NK cells against K562-CD19 cells (CD19⁺HLA-DR^{neg}, as a model of HLA-DR loss in cancer). To this end, we inoculated NSG mice with firefly luciferase-expressing K562-CD19 cells, followed by the same treatment scheme as above (FIG. 7A). In this case, we found that dual CAR-NK cells performed similarly to single CAR-NK cells. They both significantly inhibited tumor growth and prolonged mouse survival compared with unmodified NK cells (FIGS. 7E-7G). Therefore, dual CAR-NK cells are as effective as single CAR-NK cells in killing target cells expressing CD19 but not HLA-DR.

These data were thus consistent with those from in vitro assays (FIG. 4A-4C), showing that CD19⁺HLA-DR⁺double-positive cells have reduced killing by anti-CD19 CAR-NK cells bearing the anti-HLA-DR iCAR.

### Example 2

Anti-CD33 CAR-NK Cells Bearing the Anti-HLA-DR iCAR Preferentially Target HLA-DR-Negative AML Cells

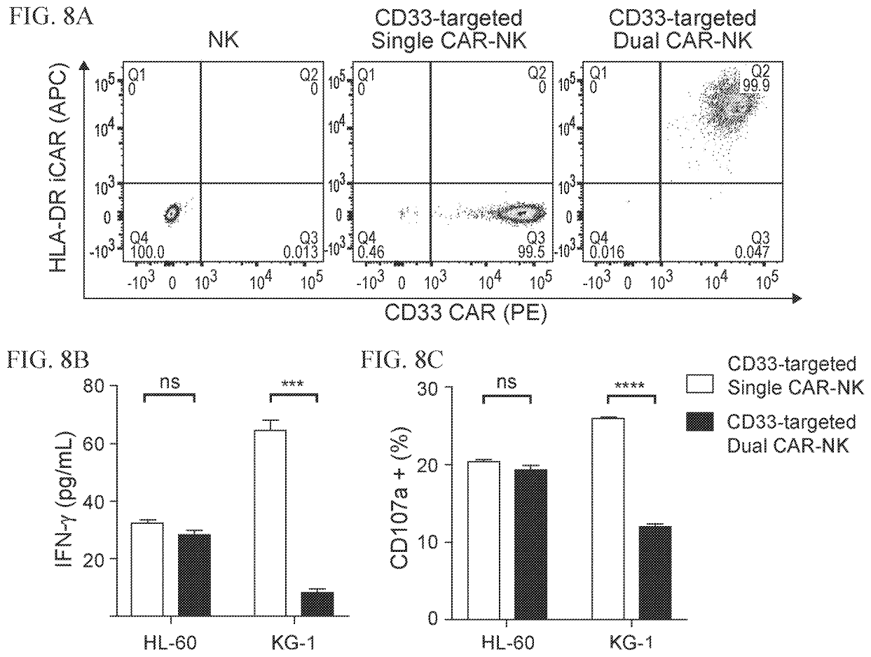

To examine the generality of the anti-HLA-DR iCAR, we additionally tested the anti-HLA-DR iCAR in combination with an anti-CD33 CAR. For this purpose, we engineered NK-92MI cells to express a CD28/CD3C-based anti-CD33 CAR with or without the anti-HLA-DR iCAR (FIG. 8A). We then compared their activation levels against HL-60 (CD33+HLA-DR^{neg}) and KG-1 cells (CD33⁺HLA-DR⁺) cells. Both IFN-γ production and CD107a degranulation assays showed that: (1) dual CAR-NK cells were as active as single CAR-NK cells towards HL-60 cells; (2) dual CAR-NK cells were significantly less active than were single CAR-NK cells towards KG-1 cells—the production of IFN-γ was reduced by more than 80%, and the percentage of CD107a⁺ population was reduced by approximately 50% (FIGS. 8B and 8C). Therefore, the anti-HLA-DR iCAR can potentially be used to enhance the anti-leukemia specificity of anti-CD33 CAR-NK cells against HLA-DR-negative AML, which has been observed at diagnosis or relapse after transplantation.

Overall, on target off-tumor toxicity presents a significant safety concern for CAR-T and CAR-NK cell therapy because target antigens are rarely cancer-specific. Since HLA-DR loss has been well documented in a substantial proportion of hematologic malignancies, we propose to enhance anti-cancer specificity through dual targeting: activating CAR-T and CAR-NK cells in response to cancer antigens while inhibiting them in response to HLA-DR. To this end, we have developed a PD-1-based anti-HLA-DR iCAR that inhibits NK-cell activation. We show that anti-CD19 or anti-CD33 CAR-NK cells bearing the anti-HLA-DR iCAR can preferentially target HLA-DR-negative cells over HLA-DR-positive cells. We also find that the level of CAR-NK cell inhibition is positively correlated with the densities of HLA-DR and iCAR. Moreover, we find that HLA-DR-expressing surrounding cells do not affect the target selectivity of dual CAR-NK cells. Thus, our approach can potentially be used to enhance the specificity of CAR-NK cell therapy against various malignancies with HLA-DR loss.

Our study makes at least two contributions: we use iCAR to target HLA-DR, a self-antigen critical to the immune system but frequently lost on malignant cells; and we demonstrate the feasibility of using iCAR to enhance the targeting specificity of CAR-NK cells. In contrast to T cells, allogeneic NK cells have reduced risk of inducing graft-versus-host diseases (GVHD). There is thus increasing interest in engineering CAR-NK cells as potential off-the-shelf cellular therapeutics. The iCAR platform can potentially further enhance the safety of CAR-NK cell therapy. While this study is focused on CAR-NK cell targeting, the anti-HLA-DR iCAR could also be incorporated in CAR-T cells. It should be noted that T cells express HLA-DR upon activation. That may or may not affect the expression or function of anti-HLA-DR iCAR in T cells.

In line with our expectation, dual CAR-NK cells preferentially recognize and kill HLA-DR-negative cells over HLA-DR-positive cells. In comparison with single CAR-NK cells, dual CAR-NK cells were equally reactive against HLA-DR-negative cells but were up to 50-80% less reactive against HLA-DR-positive cells in vitro (FIGS. 4A-4C, 5A, 5C, 6D, 8B-8C, 4D, 4E, and 6E). We also confirmed that HLA-DR expression could effectively protect target cells from cytotoxicity mediated by dual CAR-NK cells in vivo (FIGS. 7A-7G and 7H). A promising approach to improve potency and selectivity of dual CAR-NK cells is to utilize signaling domains derived from NK cells instead of T cells. NK-cell signaling differs significantly from T-cell signaling. NK cells express a series of germline-encoded activating receptors such as the NK group 2 member D receptor (NKG2D), the Fc receptor CD16 (FcγRIIIa), and 2B4 (CD244), and these receptors signal through molecules such as DAP10, FcεRIγ, and CD3ζ. Recently, Li et al. reported that NKG2D/2B4/DAP10/CD3ζ-based CARs were more effective than CD28/4-1BB/CD3ζ-based CARs in enabling NK cells to kill ovarian cancer cells. NK cells also express strong inhibitory receptors, such as the NK group 2 member A receptor (NKG2A) and the killer cell immunoglobulin-like receptors (KIR). Future research should focus on optimizing CAR and iCAR using NK cell-derived signaling domains. Another approach to enhance dual CAR-NK cells is to elevate the expression levels of anti-HLA-DR iCAR, as we observed that the level of inhibition was positively correlated with the iCAR density (FIGS. 5B and 5C). Currently, the expression of the iCAR used in our construct is controlled by the transcriptional promotor UBC1. The expression density of the iCAR could potentially be increased by using a stronger transcriptional promoter such as EF1a or MSCV.

Cancer cells might reverse HLA-DR downregulation to escape dual CAR-NK cell attack. The loss of HLA-DR expression can be reversible or irreversible depending on the mechanism of loss. HLA-DR loss via genetic deletion is irreversible. For example, HLA-DR loss in DLBCL in immune-privileged sites is mainly due to homozygous deletion of the HLA II region on chromosome 6. HLA-DR can also be lost by a combination of hemizygous deletions and stop-codon mutations in the other allele. On the other hand, HLA-DR downregulation through transcriptional regulation is reversible. For example, a common mechanism of HLA-DR downregulation is decreased expression of the MHC class II transactivator CIITA, and it is known that CIITA can be upregulated by IFN-γ. However, the expression of HLA-DR would expose cancer cells to host immune surveillance, as evidenced by increased tumor-infiltrating T cells in HLA-DR positive cases. If cancer cells can and will upregulate HLA-DR expression, a potential solution would be to combine dual CAR-NK cells with another therapy that can target HLA-DR⁺ cancer cells, e.g., donor lymphocyte infusion and TCR-based immunotherapy.

While this study is focused on targeting HLA-DR loss in hematologic malignancies, the same approach can potentially be used to target HLA class I loss in solid tumors. On-target off-tumor toxicity may prove to be problematic in the treatment of solid tumors because target antigens may be expressed in multiple organs. For example, liver toxicity was observed in a renal carcinoma clinical trial testing CAR-T cells against carbonic anhydrase IX (CAIX), which is also expressed on the epithelial cells of bile ducts. Moreover, a colon cancer patient died of respiratory failure after infusion of anti-ERBB2 CAR-T cells, probably due to off-tumor recognition of lung epithelial cells that express ERBB2 at low levels. Loss of the expression of HLA class I alleles (including HLA-A, HLA-B, and HLA-C) has been well documented in a variety of solid tumors, including up to 40-50% of breast, prostate, and lung cancers, and about 15-25% of colorectal and bladder cancers. Therefore, CAR-T and CAR-NK cells can be engineered to target malignancies with HLA class I and II loss specifically.

In summary, we have developed new dual CAR-NK cells that can kill HLA-DR-negative cells but spare HLA-DR-positive cells.

### Techniques

Cell lines and cell culture. NK-92MI cells (ATCC) were maintained in RPMI-1640 media supplemented with 20% fetal bovine serum (FBS). K562-CD19 cells were a gift from Dr. Pin Wang (USC Viterbi School of Engineering). K562-CD19-HLA-DR cells were generated by lentiviral transduction of K562-CD19 cells to express full-length HLA-DR molecules. K562, K562-CD19, K562-CD19-HLA-DR, Nalm6, KOPN1, and Raji cells were cultured in complete RPMI-1640 medium supplemented with 10% FBS. 293T cells were grown in DMEM medium supplemented with 10% FBS.

Plasmid construction. The lentiviral vector expressing the anti-CD19 CAR (pFUW-HA-antiCD19-CD28-CD3C) was a gift from Dr. Pin Wang (USC Viterbi School of Engineering). The lentiviral vector expressing the anti-HLA-DR iCAR (pFUW-FLAG-anti-HLA-DR-PD1) was constructed as follows. The anti-HLA-DR scFv in the format of VL-(GGGGS)3-VH was designed based on a full human HLA-DR antibody (1D09C3). The anti-HLA-DR iCAR consists of an N-terminal FLAG tag, an anti-HLA-DR scFv, and PD-1 hinge, transmembrane, and intracellular domain. The whole gene of iCAR was synthesized as a gBlocks gene fragment (IDT Technologies). The plasmid pFUW-FLAG-anti-HLA-DR-PD1 was constructed by inserting the iCAR gene into the pFUW linear vector (BamH I, EcoR I).

Lentiviral production and transduction. 293T cells were seeded one day before transduction to achieve 90% confluence in two 100 mm dishes (VWR). The plasmid encoding the anti-CD19 CAR or the anti-HLA-DR iCAR was mixed thoroughly with the three packaging plasmids (encoding RRE, REV, and VSVG) in 2×HEPES-buffered saline (HBS). The plasmid-containing HBS buffer was slowly added to 0.25 M CaCl₂) solution. The whole mixture was then added to the 293T cell culture from the side of the dish. After a 4-hour incubation, the medium was replaced with fresh DMEM supplemented with 10% FBS. Three days after infection, lentiviruses were harvested and filtered through a 0.45 μm filter (Pall), concentrated using 100 kD ultrafilters (Amicon), and loaded into 1×10⁶ NK-92MI cells in a 24-well untreated plate. NK cells were supplemented with 8 μg/ml protamine sulfate (Sigma-Aldrich) and 6 μM BX795 (Invivogen) to enhance transduction efficiency. Cells were then centrifuged for 90 minutes at 2,400 rpm at room temperature, followed by overnight incubation at 37° C. On the next day, cells were washed twice and supplemented with human recombinant IL-2 (BioLegend, San Diego, CA) to enhance viability.

Generation of the cell line K562-CD19-HLA-DR. The parent lentiviral vector pCDH-EF1-Nluc-P2A-copGFP-T2A-Puro was a gift from Kazuhiro Oka (Addgene plasmid #73022; n2t.net/addgene:73022; RRID: Addgene_73022). The HLA-DR alpha chain (UniProtKB-P01903) and beta chain (UniProtKB-P04229) were linked by a furin recognition sequence and a P2A peptide. The gene of HLA-DRα-Furin-P2A-HLA-DRP was synthesized as a gBlocks gene fragment (IDT Technologies) and inserted into the pCDH vector (BamH I, EcoR I). Lentiviral particles were generated by co-transfecting HEK293T cells with pCDH-EF1-HLA-DRα-Furin-P2A-HLA-DRβ and three packaging plasmids (encoding RRE, REV, and VSVG). K562-CD19 cells were then modified to express HLA-DR by lentiviral transduction. The transduced cells were stained with a PE-conjugated anti-HLA-DR antibody (BioLegend, San Diego, CA) and purified using the FACSAria Fusion Cell Sorter.

Analysis of CD19 and HLA-DR expression on target cell surfaces. Cells (1×10⁶) in FACS buffer (PBS with 2% FBS, 2 mM EDTA, 0.05% sodium azide) were stained with PE-conjugated anti-CD19 or anti-HLA-DR antibodies (BioLegend, San Diego, CA) at saturation on ice for 30 minutes. PE-conjugated mouse IgG-x (BioLegend, San Diego, CA) was used as an isotype control. Cells were washed three times, resuspended in FACS buffer, and analyzed by flow cytometry. Data were analyzed with FlowJo software (TreeStar Inc.).

Comparison of CAR and iCAR expression on single and dual CAR-NK cells. Different CAR-NK cells were stained with PE-conjugated anti-FMC63 scFv antibody (ACRO Biosystems, DE, USA) and anti-FLAG-tag antibody (BioLegend, San Diego, CA) at saturating concentrations, respectively. Cells were analyzed by flow cytometry, and data were analyzed with FlowJo software. The saturating concentration of each PE-conjugated antibody was determined by increasing the antibody concentration until there was no further increment of mean fluorescence intensity of stained cells.

Flow cytometry analysis. All antibodies were purchased from Thermo Fisher Scientific (MA, USA), BioLegend (CA, USA), and ACRO Biosystems (DE, USA). Cells were harvested from in vitro culture, washed twice with FACS buffer (PBS with 2% FBS and 2 mM EDTA), and stained with fluorophore-conjugated antibodies for 30 minutes on ice before analysis. Flow cytometry was performed on the BD LSR II analyzer (BD Biosciences). Data were analyzed using FlowJo.

Cytokine production assays. Single or dual CAR-NK cells (1×10⁵) were cocultured with target cells (1×10⁵) in a 96-well U-bottom plate. For testing the effect of surrounding cells, the indicated amount of KG1 or myeloid cells were added into the coculture at the beginning of the experiment. After a 4-hour incubation, the cell culture supernatant was harvested, and the concentration of IFN-γ in each sample was determined using a human IFN-γ ELISA kit (Invitrogen, Carlsbad, CA) in accordance with the manufacturer's instruction. The chemiluminescence was measured by Synergy H1 Hybrid Multiplate reader (BioTek). Data are presented as mean±SEM of triplicates.

CD107a degranulation assays. NK cells, single CAR-NK cells, or dual CAR-NK cells (1×10⁵) were cocultured with each of the six target cells at a 1:1 ratio in 200 pl medium with a PE-conjugated anti-CD107a antibody (BioLegend, San Diego, CA). After a 1-hour incubation, 100 μg/ml monensin (GolgiStop, BD Bioscience, San Jose, CA) was added to the co-culture medium. After a 4-hour incubation, cells were collected and washed twice using FACS buffer before staining on ice with an APC-conjugated anti-CD56 antibody (BD Bioscience, San Jose, CA) to differentiate NK cells from target cells. After washing three times, cells were resuspended in FACS buffer for flow cytometry analysis. The degranulated NK cells were identified as the CD107a⁺ population in CD56⁺ cells. Non-transduced NK cells were used as the negative control. Data were analyzed using FlowJo.

LDH cytotoxicity assays. 5×10⁴ NK cells, single CAR-NK cells, or dual CAR-NK cells were incubated with targeted cells at three different E:T ratios (0.2:1, 1:1, and 5:1) in a total volume of 100 pl for 4 hours. The supernatants were collected, and the released LDH was measured by a colorimetric reaction using Pierce LDH Cytotoxicity Assay Kit (Thermo Fisher, Rockford, IL). The chemiluminescence was measured by Synergy H1 Hybrid Multiplate reader (BioTek). Spontaneous LDH release controls of effector and target cells were included by incubating the effector and target cells alone. The maximum LDH release control of each target cell was calculated by adding the lysis buffer 45 minutes before supernatant collection. The percentage of cytotoxicity (% Cytotoxicity) was calculated by: (Experimental value−Effector Cells Spontaneous Control−Target Cells Spontaneous Control)×100/(Target Cell Maximum Control−Target Cells Spontaneous Control).

Myeloid cell isolation. Human peripheral blood mononuclear cells (PBMC) were isolated from the buffy coat (ZenBio, NC) by Ficoll-Paque (GE healthcares, IL) density gradient centrifugation at 800×g for 30 minutes at room temperature. Myeloid cells were further enriched by negative selection using Human Monocyte Enrichment Kit (STEMCELL Technologies).

Animal studies. All animal protocols and experiments were approved by the USC Institutional Animal Care and Use Committee (IACUC). 8- to 10-week-old NOD.Cg-Prkdc^{scid}Il2rg^{tm1Wjl}/SzJ (NSG) mice were purchased from The Jackson Laboratory (ME, USA). The K562-CD19-HLA-DR-Luc and K562-CD19-Luc cells were generated by transducing K562-CD19-HLA-DR and K562-CD19 cells with pCDH-EF1-Luc2-P2A-copGFP (a gift from Kazuhiro Oka; Addgene plasmid #72485; n2t.net/addgene:72485; RRID: Addgene_72485), followed by purification using the FACSAria Fusion Cell Sorter. On day 0, NSG mice were intravenously inoculated with 5×10⁵ K562-CD19-HLA-DR-Luc cells or K562-CD19-Luc cells. On day 3, the bioluminescence of the engrafted tumor cells was monitored by IVIS imaging (PerkinElmer, Waltham, MA). Mice were then randomly divided into 3 groups (n=5). Each group was treated with 1×10⁷ NK cells, single CAR-NK cells, or dual CAR-NK cells by tail vein injection on day 3. Tumor growth was monitored by bioluminescence imaging once a week. Survival data were reported in Kaplan-Meier plots and analyzed by log-rank test. Images were analyzed using Caliper Life Sciences software (PerkinElmer, Waltham, MA).

Construction, expression, refolding, and purification of the anti-HLA-DR scFv. The gene of the anti-HLA-DR scFv (clone 1D09C3) in the format of VL-(GGGGS)3-VH was inserted into a linear vector pET22b (Nde I, Xho I) to construct pET22b-anti-HLA-DR-scFv. The plasmid was transformed into E. coli BL21 (DE3) competent cells. Cells harboring the plasmid were grown in the LB medium containing 100 μg/ml ampicillin. When OD600 reached 0.5, protein expression was induced by adding 1 mM isopropyl-β-D-thiogalactopyranoside (IPTG). After 4 hours, cells were harvested by centrifugation and lysed by sonication. The inclusion body of the scFv was isolated by centrifugation and solubilized in a denaturation buffer (100 mM Tris-HCl, pH 8.0, 200 mM NaCl, 1 mM EDTA, and 6 M GuHCl). The native scFv protein was obtained by in vitro refolding using a published method. The refolded scFv was further purified by gel filtration chromatography using a Hiload Superdex 200 16/600 column by FPLC (GE Healthcare).

Statistical analysis. Data are presented as means±SEM. The survival statistics were calculated using the log-rank (Mantel-Cox) test. The other differences between groups were analyzed by unpaired two-tailed Student's t-test. The statistical significance was defined at p<0.05 (ns, p>0.05; * p<0.05; ** p<0.01; *** p<0.001; **** p<0.0001). All statistical analyses were performed using GraphPad Prism 8.0.

Amino acid sequence of anti-CD19 scFv
(clone: FMC63)(SEQ ID NO: 1):
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLEITGGGGSGGGGSGGGGSEVKLQESGPGLVA
PSQSLSVTCTVSGVSLPDYGVSWIRQPPRKGLEWLGVIWGSETTY
YNSALKSRLTIIKDNSKSQVFLKMNSLQTDDTAIYYCAKHYYYGG
SYAMDYWGQGTSVTVSS
VL sequence (SEQ ID NO: 2):
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLEIT
VH sequence (SEQ ID NO: 3):
EVKLQESGPGLVAPSQSLSVTCTVSGVSLPDYGVSWIRQPPRKGL
EWLGVIWGSETTYYNSALKSRLTIIKDNSKSQVFLKMNSLQTDDT
AIYYCAKHYYYGGSYAMDYWGQGTSVTVSS
Amino acid sequence of Anti-CD33 scFv
(clone: my96)(SEQ ID NO: 4):
EIVLTQSPGSLAVSPGERVTMSCKSSQSVFFSSSQKNYLAWYQQI
PGQSPRLLIYWASTRESGVPDRFTGSGSGTDFTLTISSVQPEDLA
IYYCHQYLSSRTFGQGTKLEIKRGGGGSGGGGSSGGGSQVQLQQP
GAEVVKPGASVKMSCKASGYTFTSYYIHWIKQTPGQGLEWVGVIY
PGNDDISYNQKFQGKATLTADKSSTTAYMQLSSLTSEDSAVYYCA
REVRLRYFDVWGQGTTVTVSS
VL sequence (SEQ ID NO: 5)
EIVLTQSPGSLAVSPGERVTMSCKSSQSVFFSSSQKNYLAWYQQI
PGQSPRLLIYWASTRESGVPDRFTGSGSGTDFTLTISSVQPEDLA
IYYCHQYLSSRTFGQGTKLEIKR
VH sequence (SEQ ID NO: 6)
QVQLQQPGAEVVKPGASVKMSCKASGYTFTSYYIHWIKQTPGQGL
EWVGVIYPGNDDISYNQKFQGKATLTADKSSTTAYMQLSSLTSED
SAVYYCAREVRLRYFDVWGQGTTVTVSS
Amino acid sequence of Anti-HLA-DR scFv
(clone: 1D09C3)(SEQ ID NO: 7):
DIVLTQPPSVSGAPGQRVTISCSGSESNIGNNYVQWYQQLPGTAP
KLLIYDNNQRPSGVPDRFSGSKSGTSASLAITGLQSEDEADYYCQ
SYDMNVHVFGGGTKLTVLGGGGGSGGGGSGGGGSQVQLKESGPAL
VKPTQTLTLTCTFSGFSLSTSGVGVGWIRQPPGKALEWLALIDWD
DDKYYSTSLKTRLTISKDTSKNQVVLTMTNMDPVDTATYYCARSP
RYRGAFDYWGQGTLVTVSS
VL sequence (SEQ ID NO: 8):
DIVLTQPPSVSGAPGQRVTISCSGSESNIGNNYVQWYQQLPGTAP
KLLIYDNNQRPSGVPDRFSGSKSGTSASLAITGLQSEDEADYYCQ
SYDMNVHVFGGGTKLTVLG
VH sequence (SEQ ID NO: 9):
QVQLKESGPALVKPTQTLTLTCTFSGFSLSTSGVGVGWIRQPPGK
ALEWLALIDWDDDKYYSTSLKTRLTISKDTSKNQVVLTMTNMDPV
DTATYYCARSPRYRGAFDYWGQGTLVTVSS

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A genetically engineered immune cell expressing a first chimeric antigen receptor (CAR) and a second, inhibitory CAR, by introduction of one or more genes, wherein the first CAR comprises:
a. a first antigen-specific binding domain,
b. a first transmembrane domain, and
c. a first intracellular domain;
and the second, inhibitory CAR comprises:
a. a second antigen-specific binding domain, which is specific for and binds a human leukocyte antigen (HLA),
b. a second transmembrane domain, and
c. a second intracellular domain, which is a signaling domain or intracellular tail of an immunoinhibitory receptor.

2. The genetically engineered immune cell of claim 1, which is a natural killer (NK) cell or a T cell, and the genetically engineered immune cell is more selective in targeting a malignant cell or tumor cell that expresses an antigen that the first antigen-specific binding domain binds to but does not express the HLA, than targeting a malignant cell or tumor cell that expresses both the antigen that the first antigen-specific binding domain binds to and the HLA.

3. The genetically engineered immune cell of claim 1, wherein the second antigen-specific binding domain is specific for and binds HLA-DR, and the genetically engineered immune cell is an NK cell that does not express HLA-DR.

4. The genetically engineered immune cell of claim 1, wherein the second antigen-specific binding domain comprises:
a single-chain variable fragment (scFv) having the sequence as set forth in SEQ ID NO:7 or at least 90% identical to SEQ ID NO:7, or
a variable region of the light chain (VL) having the sequence as set forth in SEQ ID NO:8 or at least 90% identical to SEQ ID NO:8, and a variable region of the heavy chain having the sequence as set forth in SEQ ID NO:9 or at least 90% identical SEQ ID NO:9, and a linker between the VL and the VH.

5. The genetically engineered immune cell of claim 1, wherein the second antigen-specific binding domain comprises:
a single-chain variable fragment (scFv) from anti-HLA-DR monoclonal antibody clone L243.

6. The genetically engineered immune cell of claim 1, wherein the second intracellular domain is the signaling domain or intracellular tail of an immunoinhibitory receptor selected from the group consisting of programmed cell death protein 1 (PD-1), cytotoxic T lymphocyte-associated antigen-4 (CTLA-4), lymphocyte-activation gene 3 (LAG-3), 2B4, B- and T-lymphocyte attenuator (BTLA), and a combination thereof; optionally the second intracellular domain being the signaling domain or intracellular tail of PD-1.

7. (canceled)

8. The genetically engineered immune cell of claim 6, wherein the first antigen-specific binding domain is specific for and binds an antigen selected from the group consisting of antigens specific for cancer, an inflammatory disease, a neuronal disorder, diabetes, a cardiovascular disease, an infectious disease, an autoimmune disease, and a combination thereof; optionally the first antigen-specific binding domain being specific for CD33 or specific for CD19.

9. (canceled)

10. The genetically engineered immune cell of claim 1, wherein the first antigen-specific binding domain comprises:
a single-chain variable fragment (scFv) having the sequence as set forth in SEQ ID NO:4 or at least 90% identical to SEQ ID NO:4, or
a variable region of the light chain (VL) having the sequence as set forth in SEQ ID NO:5 or at least 90% identical to SEQ ID NO:5, and a variable region of the heavy chain having the sequence as set forth in SEQ ID NO:6 or at least 90% identical SEQ ID NO:6, and a linker between the VL and the VH.

11. (canceled)

12. The genetically engineered immune cell of claim 1, wherein the first antigen-specific binding domain comprises:
a single-chain variable fragment (scFv) having the sequence as set forth in SEQ ID NO:1 or at least 90% identical to SEQ ID NO:1, or
a variable region of the light chain (VL) having the sequence as set forth in SEQ ID NO:2 or at least 90% identical to SEQ ID NO:2, and a variable region of the heavy chain having the sequence as set forth in SEQ ID NO:3 or at least 90% identical SEQ ID NO:3, and a linker between the VL and the VH.

13. The genetically engineered immune cell of claim 1, wherein the first transmembrane domain comprises CD28 transmembrane domain, CD8 transmembrane domain, a transmembrane domain of a zeta chain of a T cell receptor complex, or a combination thereof; and
wherein the second transmembrane domain comprises a transmembrane domain of the immunoinhibitory receptor, CD28 transmembrane domain, CD8 transmembrane domain, a transmembrane domain of a zeta chain of a T cell receptor complex, or a combination thereof; and
wherein the first intracellular domain comprises one or more of a CD3 zeta chain, FcγRIII, FcεRI, a cytoplasmic tail of a Fc receptor, and an immunoreceptor tyrosine-based activation motif (ITAM) bearing cytoplasmic receptors, and optionally further comprising a co-stimulatory domain selected from the group consisting of CD28, CD137 (4-1BB), CD134 (OX40), Dap10, CD27, CD2, CD5, ICAM-1, LFA-1, Lck, TNFR-I, TNFR-II, Fas, CD30, CD40 and a combination thereof.

14. The genetically engineered immune cell of claim 1, wherein the first CAR further comprises a first extracellular spacer domain, positioned between the first antigen-specific binding domain and the first transmembrane domain, and the first extracellular spacer domain comprises (i) a hinge region of CD8α, (ii) a hinge region of IgG4, (iii) a hinge and CH2 region of IgG4, (iv) a hinge, CH2 and CH3 region of IgG4, (v) a hinge, CH2 and CH3 region of IgG1, (vi) a hinge region of IgG1, (vi) a hinge and CH2 region of IgG1, or (vii) a combination of any two or more of (i)-(vi); and/or
wherein the second, inhibitory CAR further comprises a second extracellular spacer domain, positioned between the second antigen-specific binding domain and the second transmembrane domain, and the second extracellular spacer domain comprises (1) an extracellular topological domain of the immunoinhibitory receptor, (2) a hinge region of CD8α, (3) a hinge region of IgG4, (4) a hinge and CH2 region of IgG4, (5) a hinge, CH2 and CH3 region of IgG4, (6) a hinge, CH2 and CH3 region of IgG1, (7) a hinge region of IgG1, (8) a hinge and CH2 region of IgG1, or (9) a combination of any two or more of (1)-(8); and/or
wherein the first CAR, the second, inhibitory CAR, or both, further comprises a protein tag at N terminus; and the protein tag of the first CAR and that of the second, inhibitory CAR are different, if both are present.

15. (canceled)

16. (canceled)

17. The genetically engineered immune cell of claim 1, whose secretion level of IFN-γ, after contact of the genetically engineered immune cell with a target cell expressing an antigen that the first antigen-specific binding domain is specific for and binds to, and/or whose cytotoxicity level towards the target cell expressing the antigen that the first antigen-specific binding domain is specific for and binds to, is statistically similar to that of a genetically engineered immune cell that is otherwise identical except for lacking the second, inhibitory CAR; and/or
whose secretion level of the IFN-γ after the contact with the target cell, whose degranulation if the genetically engineered immune cell is an NK cell upon incubation with a second target cell expressing the HLA, and/or whose cytotoxicity level towards the second target cell expressing the HLA, is at least 80%, 70%, 60%, 50%, 40%, 30%, or 20% lower, or at least two-fold lower, than the genetically engineered immune cell that is otherwise identical except for lacking the second, inhibitory CAR.

18. (canceled)

19. The genetically engineered immune cell of claim 1, wherein the second, inhibitory CAR is encoded by a coding sequence in a gene construct introduced to the genetically engineered immune cell, and the coding sequence encoding the second, inhibitory CAR is operably linked to a transcriptional promoter comprising UBC1, EF1α or MSCV.

20. A combination, comprising:
a first genetically engineered immune cell expressing a first chimeric antigen receptor (CAR), wherein the first CAR comprises:
a. a first antigen-specific binding domain,
b. a first transmembrane domain, and
c. a first intracellular domain;
and
a second genetically engineered immune cell expressing a second, inhibitory CAR, wherein the second, inhibitory CAR comprises:
a. a second antigen-specific binding domain, which is specific for and binds a human leukocyte antigen (HLA),
b. a second transmembrane domain, and
c. a second intracellular domain, which is a signaling domain or intracellular tail of an immunoinhibitory receptor.

21. A genetically engineered natural killer (NK) cell expressing an inhibitory chimeric antigen receptor (CAR) by introduction of a gene, wherein the inhibitory CAR comprises:
a. an antigen-specific binding domain, which is specific for and binds human leukocyte antigen DR (HLA-DR),
b. a transmembrane domain, and
c. an intracellular domain, which is a signaling domain or intracellular tail of an immunoinhibitory receptor of PD-1.

22. A vector encoding the second, inhibitory CAR of claim 1.

23. A method of preparing a genetically engineered immune cell of claim 1, comprising introducing to an immune cell a first vector encoding the first CAR and a second vector encoding the second, inhibitory CAR.

24. (canceled)

25. The method of claim 23, wherein the first vector and the second vector are introduced sequentially, and the method further comprises selecting the immune cell expressing the first CAR after the first vector is introduced to the immune cell, wherein the second vector is introduced to the selected immune cell expressing the first CAR, so as to produce the genetically engineered immune cell expressing both the first CAR and the second, inhibitory CAR.

26. A method of treating a subject with a hematologic malignancy or a solid tumor, comprising:
administering to the subject a pharmaceutical composition comprising an effective quantity of genetically engineered immune cells of claim 1; optionally the subject is detected or diagnosed with complete or partial loss of HLA-DR in the hematologic malignancy or detected or diagnosed with complete or partial loss of HLA-A, HLA-B and/or HLA-C in the solid tumor.

27. (canceled)

28. The method of claim 26, wherein the subject has a hematologic malignancy and is detected or diagnosed with complete or partial loss of HLA-DR in the hematologic malignancy, and the hematologic malignancy comprises acute myeloid leukemia (AML), classical Hodgkin lymphoma (CHL), chronic myelomonocytic leukemia (CMML), chronic myeloid leukemia (CML), or a combination thereof; optionally the subject being an AML patient with post-transplant relapse.

29. (canceled)

30. The method of claim 28, wherein the pharmaceutical composition is administered to bloodstream or bone marrow of the subject.

31. The method of claim 26, wherein the genetically engineered immune cells are produced by introducing the one or more genes encoding the first CAR and the second, inhibitory CAR into allogeneic NK cells obtained from another subject.

32. The method of claim 26, wherein the subject is detected or diagnosed with complete or partial loss of HLA-A, HLA-B and/or HLA-C in the solid tumor, and the method further comprising administering to the subject a T-cell receptor-based immunotherapy or donor lymphocyte infusion, so as to target cancer cells that express the HLA.
